(19) United States
(12) Patent Application Publication
(10) Pub. No.: US 2025/0319073 A1
(43) Pub. Date: Oct. 16, 2025

(54) COMPOSITIONS AND METHODS FOR THE TREATMENT OF HUNTINGTON’S DISEASE AND HTT PROTEINOPATHIES

(71) Applicant: Stealth BioTherapeutics Inc., Needham, MA (US)

(72) Inventor: Dennis Keefe, Bedford, MA (US)

(73) Assignee: Stealth BioTherapeutics Inc., Needham, MA (US)

(21) Appl. No.: 18/866,313

(22) PCT Filed: May 16, 2023
(86) PCT No.: PCT/US2023/022375

§ 371 Date: Nov. 15, 2024

Related U.S. Application Data
(60) Provisional application No. 63/343,230, filed on May 18, 2022.

(51) Int. Cl. A61K 31/4245; A61K 9/00; A61K 45/06; A61P 25/28

(52) U.S. Cl. CPC A61K 31/4245; A61K 9/0019; A61K 45/06; A61P 25/28

(57) ABSTRACT

The present disclosure provides compounds, compositions, and/or methods for treating, preventing, inhibiting, amelio-rating, or delaying the onset of Huntington's disease and/or a HTT proteinopathy in a subject. The methods can comprise administering to the subject an effective amount of a peptidomimetic compound, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5- guanidinopentanamide, or a pharmaceutically acceptable salt, stereoisomer, tautomer, hydrate, and/or solvate thereof, alone or in combination with one or more other therapeutic agents.

### CROSS-REFERENCE TO RELATED APPLICATIONS

This application claims the benefit of and priority to U.S. Provisional Patent Application No. 63/343,230, filed May 18, 2022, the entire contents of which are incorporated herein by reference.

### TECHNICAL FIELD

The present technology relates generally to compounds, compositions/medicaments and/or methods for treating, preventing, inhibiting, ameliorating and/or delaying the onset of Huntington's disease and/or a HTT proteinopathy. Additionally, the present technology relates to administering an effective amount of a peptidomimetic compound, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof, to a subject suffering from, or at risk for, Huntington's disease and/or a HTT proteinopathy.

### INTRODUCTION

The following description is provided to assist the understanding of the reader. None of the information provided or references cited is admitted to be prior art to the compositions and methods disclosed herein.

Neurodegenerative disease and disorders affect a subject's activities such as balance, movement, talking, breathing and/or heart function. Neurodegenerative disease and disorders are generally incurable and debilitating conditions that result in progressive degeneration and/or death of nerve cells. In many cases, the neurodegenerative disease is often directly or indirectly attributable to the death of the subject. Typically, there are drugs available to address symptoms but all too often there are no treatments that curtail the progression or severity of the disease or disorder itself. The present specification is directed to Huntington's disease and/or a HTT proteinopathy.

Huntington's disease is a progressive neurodegenerative disorder that causes motor impairment, cognitive decline and behavioral modifications. Huntington's disease is characterized as a proteinopathy, associated with aggregates of huntingtin protein (HTT) in the brain. Though the pathology of the disease involves accumulation of huntingtin protein in the brain, Huntington's disease is a genetic autosomal dominate disorder caused by a mutation in the gene HTT that results in a CAG repeat expansion. The age of onset and aggressiveness of the disease appears to closely correlate with the length of the CAG repeat expansion (Lee et al., Neurology (2012) 78: 690-695). The repeat expansion appears to increase during the lifetime of the subject (Kacher et al. eLife (2021) 10: e64674). The disease often becomes symptomatic between the ages of 30 to 50 but can occur at anytime, even in early childhood (at age 2 or up) and at advanced age (e.g. >70). The average lifespan after diagnosis of Huntington's disease is about 10 to 30 years, with an average of about 15-17 years. No effective treatments for Huntington's disease are available. Currently available medications are directed to treating or ameliorating symptoms. Accordingly, there is a need in the art to develop treatment options for subjects with Huntington's disease and/or a HTT proteinopathy.

### SUMMARY

In one aspect, the present disclosure provides a method for treating, preventing, inhibiting, ameliorating or delaying the onset of Huntington's disease and/or a HTT proteinopathy in a subject in need thereof, comprising administering to the subject a therapeutically effective amount of a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof. In some embodiments, the peptidomimetic is a peptidomimetic of Formula II, or a pharmaceutically acceptable salt, stereoisomer, tautomer, hydrate, and/or solvate thereof. In some embodiments, the peptidomimetic is a peptidomimetic of Formula I or Ia, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, the subject has been diagnosed as having Huntington's disease. In some embodiments, the subject has been diagnosed as having a HTT proteinopathy.

In some embodiments, the peptidomimetic is administered to the subject daily for 2 weeks or more, for 12 weeks or more, for 24 weeks or more, for 36 weeks or more, for 48 weeks or more, or for 52 weeks or more. In some embodiments, once diagnosed, the peptidomimetic is administered daily for the remainder of the life of the subject.

In some embodiments, the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

In some embodiments, the subject is a mammal. In some embodiments, the mammalian subject is a human.

In some embodiments, the peptidomimetic is administered orally. In some embodiments, the peptidomimetic is administered subcutaneously. In some embodiments, the peptidomimetic is administered topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the method further comprises separately, sequentially, or simultaneously administering an additional treatment to the subject. In some embodiments, the additional treatment comprises administration of a therapeutic agent. In some embodiments, the therapeutic agent is selected from the group consisting of: Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine). In some embodiments, the therapeutic agent is elamipretide (also known as SS-31 or bendavia). In some embodiments, the combination of peptidomimetic and an additional therapeutic treatment has a synergistic effect in the prevention or treatment of Huntington's disease and/or a HTT proteinopathy.

In some embodiments, the pharmaceutically acceptable salt of the peptidomimetic comprises a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri- (tris-) acid salt). In some embodiments, pharmaceutically acceptable salt comprises a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

In one aspect, the present disclosure provides a use of a composition in the preparation of a medicament for treating, preventing, inhibiting, ameliorating or delaying the onset of Huntington's disease and/or a HTT proteinopathy in a subject in need thereof, wherein the composition comprises a therapeutically effective amount of a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof. In some embodiments, the peptidomimetic is a peptidomimetic of Formula II, or a pharmaceutically acceptable salt, stereoisomer, tautomer, hydrate, and/or solvate thereof. In some embodiments, the peptidomimetic is a peptidomimetic of Formula I or Ia, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, the subject has been diagnosed as having Huntington's disease. In some embodiments, the subject has been diagnosed as having a HTT proteinopathy.

In some embodiments, the medicament is administered to the subject daily for 2 weeks or more, for 12 weeks or more, for 24 weeks or more, for 36 weeks or more, for 48 weeks or more, or for 52 weeks or more. In some embodiments, the medicament is administered daily for the remainder of the life of the subject.

In some embodiments, the use of the medicament for the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

In some embodiments, the subject is a mammal. In some embodiments, the mammalian subject is a human.

In some embodiments, the medicament is formulated for oral administration. In some embodiments, the medicament is formulated for subcutaneous administration. In some embodiments, the medicament is formulated for administration, topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the medicament is separately, sequentially, or simultaneously used with an additional treatment. In some embodiments, the additional treatment comprises use of a therapeutic agent. In some embodiments, the therapeutic agent is selected from the group consisting of. Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine). In some embodiments, the therapeutic agent is elamipretide (also known as SS-31 or bendavia). In some embodiments, the combination of medicament and an additional treatment has a synergistic effect in the prevention or treatment of Huntington's disease and/or a HTT proteinopathy.

In some embodiments, the pharmaceutically acceptable salt comprises a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri- (tris-) acid salt). In some embodiments, pharmaceutically acceptable salt comprises a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

In still another aspect, the present disclosure provides a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof, for use in treating, preventing, inhibiting, ameliorating and/or delaying the onset of Huntington's disease and/or a HTT proteinopathy in a subject in need thereof. The peptidomimetic can be used alone or as formulated in a medicament. In some embodiments, the peptidomimetic is a peptidomimetic of Formula II, or a pharmaceutically acceptable salt, stereoisomer, tautomer, hydrate, and/or solvate thereof. In some embodiments, the peptidomimetic is a peptidomimetic of Formula I or Ia, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, the subject has been diagnosed as having Huntington's disease. In some embodiments, the subject has been diagnosed as having a HTT proteinopathy.

In some embodiments, the peptidomimetic (alone or as formulated into a medicament) is administered to the subject daily for 2 weeks or more, for 24 weeks or more, for 36 weeks or more, for 48 weeks or more, or for 52 weeks or more. In some embodiments, the peptidomimetic is administered daily for the remainder of the life of the subject.

In some embodiments, the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

In some embodiments, the subject is a mammal. In some embodiments, the mammalian subject is a human.

In some embodiments, the peptidomimetic is formulated for administration orally. In some embodiments, the peptidomimetic is formulated for administration subcutaneously. In some embodiments, the peptidomimetic is formulated for administration topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the peptidomimetic is separately, sequentially, or simultaneously used with an additional treatment. In some embodiments, the additional treatment comprises use of a therapeutic agent. In some embodiments, the therapeutic agent is selected from the group consisting of: Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine). In some embodiments, the therapeutic agent is elamipretide (also known as SS-31 or bendavia). In some embodiments, the combination of medicament and an additional treatment has a synergistic effect in the prevention or treatment of Huntington's disease and/or a HTT proteinopathy.

In some embodiments, the pharmaceutically acceptable salt comprises a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri- (tris-) acid salt). In some embodiments, pharmaceutically acceptable salt comprises a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

In still one more aspect, the present disclosure provides a formulation or medicament comprising a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof, for use in treating, preventing, inhibiting, ameliorating and/or delaying the onset of Huntington's disease and/or a HTT proteinopathy in a subject in need thereof. In some embodiments, the peptidomimetic is a peptidomimetic of Formula II, or a pharmaceutically acceptable salt, stereoisomer, tautomer, hydrate, and/or solvate thereof. In some embodiments, the peptidomimetic is a peptidomimetic of Formula I or Ia, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, the subject has been diagnosed as having Huntington's disease. In some embodiments, the subject has been diagnosed as having a HTT proteinopathy.

In some embodiments, the formulation or medicament is administered to the subject daily for 2 weeks or more, for 24 weeks or more, for 36 weeks or more, for 48 weeks or more, or for 52 weeks or more. In some embodiments, the formulation or medicament is administered daily for the remainder of the life of the subject.

In some embodiments, the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

In some embodiments, the subject is a mammal. In some embodiments, the mammalian subject is a human.

In some embodiments, the formulation or medicament is administered orally. In some embodiments, the formulation or medicament is administered subcutaneously. In some embodiments, the formulation or medicament is administered topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the use of the formulation or medicament further comprises separately, sequentially, or simultaneously administering an additional treatment to the subject. In some embodiments, the additional treatment comprises administration of a therapeutic agent. In some embodiments, the therapeutic agent is selected from the group consisting of: Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine). In some embodiments, the therapeutic agent is elamipretide (also known as SS-31 or bendavia). In some embodiments, the combination of formulation or medicament and the additional treatment has a synergistic effect in the prevention or treatment of Huntington's disease and/or a HTT proteinopathy.

In some embodiments, the pharmaceutically acceptable salt comprises a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri- (tris-) acid salt). In some embodiments, pharmaceutically acceptable salt comprises a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

### BRIEF DESCRIPTION OF THE DRAWINGS

FIG. 1 is an illustrated summary of the study design of the mouse study described in Example 1. This study evaluated the effects of administration of Compound (Ia) on and R6/2 transgenic mice.

FIG. 2 is a graph of the body weights (g) of the mice studied as described in Example 1 (body weights (g) of R6/2 mice treated with Compound (Ia)). Data are presented as mean±SEM. * P<0.05, R6/2 Vehicle vs. WT Vehicle.

FIG. 3 is a bar graph summarizing data of the motor performance in a rotarod assay for mice studied as described in Example 1 (latency of the R6/2 mice treated with Compound (Ia)). Data are presented as mean±SEM. **** P<0.001, R6/2 Vehicle vs. WT Vehicle; * P<0.05, R6/2 5 mg/kg Compound (Ia) vs. R6/2 Vehicle.

FIG. 4 is a bar graph summarizing data for the total distance traveled by mice studied as described in Example 1 (total distance traveled in the open field by R6/2 mice treated with Compound (Ia)). Data are presented as mean±SEM. **** P<0.001, R6/2 Vehicle vs. WT Vehicle; * P<0.05, R6/2 5 mg/kg Compound (Ia) vs. R6/2 Vehicle.

FIG. 5 is a bar graph summarizing data for total rearing frequency in the open field for mice studied as described in Example 1 (total rearing frequency in the open field of R6/2 mice treated with Compound (Ia)). Data are presented as mean±SEM. **** P<0.0001, R6/2 Vehicle vs. WT Vehicle.

FIG. 6 is a bar graph summarizing data for ¹⁸F-FDG uptake in mice studied as described in Example 1. Data are presented as mean±SEM. Data from the following groups are presented from left to right for each tissue: WT Vehicle, R6/2 Vehicle, and R6/2 5 mg/kg Compound (Ia). *P<0.05, R6/2 Vehicle vs. WT Vehicle. **P<0.01, R6/2 5 mg/kg Compound (Ia) vs. R6/2 Vehicle. The tissues (from left to right along the x-axis) are: Amygdala L, Amygdala R, Basal Forebrain Septum, Brain Stem, Central Gray, Cerebellum, Cortex, Hippocampus L, Hippocampus R, Hypothalamus, Inferior Colliculi L, Inferior Colliculi R, Midbrain L, Midbrain R, Olfactory Bulb, Striatum L, Striatum R, Superior Colliculi, Thalamus, and Whole Brain.

FIG. 7 is a bar graph summarizing flow cytometry data for the MitoSOX analysis of reactive oxygen species (ROS) formation in Peripheral Blood Mononuclear Cells (PBMC) of mice studied as described in Example 1. Data are presented as mean±SEM. ** P<0.01, R6/2 Vehicle vs. heat stressed cells, unpaired Welch t-test.

FIG. 8 is a bar graph summarizing data for ATP generation in PBMCs of mice studied as described in Example 1. Data are presented as mean±SEM. Statistical significance: ** P<0.01, R6/2 Vehicle vs. heat stressed cells, unpaired Welch t-test.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (TRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

### I. Chemical Definitions

Definitions of specific functional groups and chemical terms are described in more detail below. The chemical elements are identified in accordance with the Periodic Table of the Elements, GAS version, Handbook of Chemistry and Physics, 7Sh Ed., inside cover. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

The abbreviations used herein have their conventional meaning within the chemical and biological arts. The chemical structures and formulae set forth herein are intended to comply with the standard rules of chemical valency known in the chemical arts. When a range of values is listed, it is intended to encompass each value and subrange within the range. For example “C1-C6 alkyl” is intended to encompass, C1, C2, C3, C4, C5, C6, C1-C6, C1-C5, C₁-C₄, C1-C3, C1-C2, C2-C6, C2-C5, C2-C4, C2-C3, C3-C5, C3-C4, C4-C6, C4-C5, and C5-C6 alkyl.

Certain compounds of the present application can exist in unsolvated forms as well as solvated forms, including hydrated forms. Solvated forms can exist, for example, because it is difficult or impossible to remove all the solvent from the compound post synthesis. In general, the solvated forms are equivalent to unsolvated forms and are encompassed within the scope of the present application. Certain compounds of the present application may exist in multiple crystalline or amorphous forms. Certain compounds of the present application may exist in various tautomeric forms. Certain compounds of the present application may exist in various salt forms. In general, all physical forms are equivalent for the uses contemplated by the present application and are intended to be within the scope of the present disclosure.

As used herein, the term “amino acid” includes both a naturally occurring amino acid and a non-natural amino acid. The term “amino acid,” unless otherwise indicated, includes both isolated amino acid molecules (i.e., molecules that include both, an amino-attached hydrogen and a carbonyl carbon-attached hydroxyl) and residues of amino acids (i.e., molecules in which either one or both an amino-attached hydrogen or a carbonyl carbon-attached hydroxyl are removed). The amino group can be alpha-amino group, beta-amino group, etc. For example, the term “amino acid alanine” can refer either to an isolated alanine H—Ala-OH or to any one of the alanine residues H—Ala-, —Ala-OH, or —Ala-. Unless otherwise indicated, all amino acids found in the compounds described herein can be either in D or L configuration. An amino acid that is in D configuration may be written such that “D” precedes the amino acid abbreviation. For example, “D-Arg” represents arginine in the D configuration. The term “amino acid” includes salts thereof, including pharmaceutically acceptable salts. Any amino acid can be protected or unprotected. Protecting groups can be attached to an amino group (for example alpha-amino group), the backbone carboxyl group, or any functionality of the side chain. As an example, phenylalanine protected by a benzyloxycarbonyl group (Z) on the alpha-amino group would be represented as Z-Phe-OH.

With the exception of the N-terminal amino acid, all abbreviations of amino acids (for example, Phe) in this disclosure stand for the structure of NH—C(R)(R′)—CO—, wherein R and R′ each is, independently, hydrogen or the side chain of an amino acid (e.g., R=benzyl and R′═H for Phe). Accordingly, phenylalanine is H-Phe-OH. The designation “OH” for these amino acids, or for peptides (e.g., Lys-Val-Leu-OH) indicates that the C-terminus is the free acid. The designation “NH₂” in, for example, Phe-D-Arg-Phe-Lys-NH₂ indicates that the C-terminus of the protected peptide fragment is amidated. Further, certain R and R′, separately, or in combination as a ring structure, can include functional groups that require protection during the liquid phase or solid phase synthesis.

Where the amino acid has isomeric forms, it is the L form of the amino acid that is represented unless otherwise explicitly indicated as D form, for example, D-Arg. Notably, many amino acid residues are commercially available in both D- and L-form. For example, D-Arg is a commercially available D-amino acid.

A capital letter “D” used in conjunction with an abbreviation for an amino acid residue refers to the D-form of the amino acid residue.

The term “DMT” refers to 2,6-di(methyl)tyrosine (e.g., 2,6-dimethyl-L-tyrosine; CAS 123715-02-6).

As used herein, the term “hydrate” refers to a compound which is associated with water. The number of the water molecules contained in a hydrate of a compound may be (or may not be) in a definite ratio to the number of the compound molecules in the hydrate.

As used herein, the term “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt3), trimethylamine, tripropylamine, tromethamine and the like, such as where the salt includes the protonated form of the organic base (e.g., [HNEt3]+). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present application may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

As used herein, the term “peptidomimetic” refers to a compound of Formula (II):

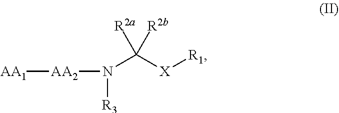

or a pharmaceutically acceptable salt, stereoisomer, tautomer, hydrate, and/or solvate thereof, as more fully described and/or claimed in WIPO published application: WO2019/118878 (See below for a definition of variables AA1, AA2, R1, R2a, R2b, R3 and X). In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Compound I, as illustrated below as Formula I), or a pharmaceutically acceptable salt (e.g. Compound (Ia), illustrated below as a tris-HCl salt as Formula (Ia)), stereoisomer, tautomer, hydrate, and/or solvate thereof.

As used herein, the term “small molecule” refers to any organic compound that affects a biologic process with a molecular weight less than 900 daltons. It is to be understood that for purposes of this definition, the molecular mass is calculated without reference to any associated (i.e. non-covalently bonded) molecules such as salts, water or other solvent molecules. As used herein, a “small molecule peptidomimetic” is a peptidomimetic with a free-base molecular weight less than 900 daltons. An example of such a small molecule peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (CAS #2356106-71-1; free-base molecular weight of 607.76) or a pharmaceutically acceptable salt, stereoisomer, tautomer, hydrate, and/or solvate thereof.

As used herein, the term “solvate” refers to forms of the compound that are associated with a solvent, possibly by a solvolysis reaction. This physical association may include hydrogen bonding. Conventional solvents include water, methanol, ethanol, isopropanol, acetic acid, ethyl acetate, acetone, hexane(s), DMSO, THF, diethyl ether, and the like.

As used herein, the term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

As used herein, the terms “(R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide,” “(D-Arg-DMT-NH((S)-5-amino-1-(3-benzyl-1,2,4-oxadiazol-5-yl)pent-1-yl),”, (2R)-2-amino-N-[(1S)-1-{[(1S)-5-amino-1-(3-benzyl-1,2,4-oxadiazol-5-yl)pentyl]carbamoyl}-2-(4-hydroxy-2,6-dimethylphenyl)ethyl]-5-carbamimidamidopentanamide, refer to the same small molecule peptidomimetic, are used interchangeably herein, and refer to a compound of the following Formula (I):

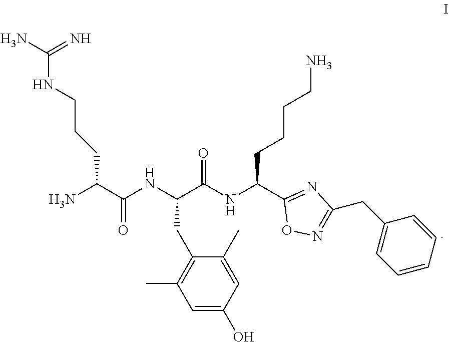 [SMILES: Cc1cc(O)cc(C)c1C[C@H](NC(=O)[C@H](N)CCCNC(=N)N)C(=O)N[C@@H](CCCCN)c1nc(Cc2ccccc2)no1]

### (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide

The term “(R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide,”, (2R)-2-amino-N-[(1S)-1-{[(1S)-5-amino-1-(3-benzyl-1,2,4-oxadiazol-5-yl)pentyl]carbamoyl}-2-(4-hydroxy-2,6-dimethylphenyl)ethyl]-5-carbamimidamidopentanamide, “(D-Arg-DMT-NH((S)-5-amino-1-(3-benzyl-1,2,4-oxadiazol-5-yl)pent-1-yl),” is intended to include pharmaceutically acceptable salt forms thereof such as the tri- (or tris)-HCl salt of Formula (Ia):

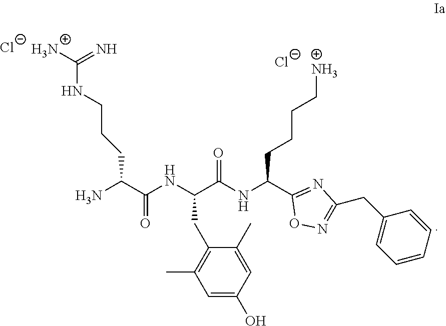 [SMILES: [Cl-] ; Cc1cc(O)cc(C)c1C[C@H](NC(=O)[C@H](N)CCCNC(=N)N)C(=O)N[C@@H](CCCC[NH3+])c1nc(Cc2ccccc2)no1 ; [Cl-]]

### II. Other Definitions

It is to be appreciated that certain aspects, modes, embodiments, variations and features of the technology are described below in various levels of detail in order to provide a substantial understanding of the present application. The definitions of certain terms as used in this specification are provided below. Unless defined otherwise, all technical and scientific terms used herein generally have the same meaning as commonly understood by one of ordinary skill in the art to which this technology belongs.

As used in this specification and the appended embodiments, the singular forms “a”, “an” and “the” include plural references unless the content clearly dictates otherwise. For example, reference to “a cell” includes a combination of two or more cells, and the like.

As used herein, “administering” or the “administration” of an agent (i.e. therapeutic agent) or compound/drug product (including a composition) to a subject includes any route of introducing or delivering to a subject a compound/drug product to perform its intended function. Administration may be carried out by any suitable route, such as oral administration. Administration can be carried out subcutaneously. Administration can be carried out intravenously. Administration can be carried out intraocularly. Administration can be carried out systemically. Alternatively, administration may be carried out topically, intranasally, intraperitoneally, intradermally, ophthalmically, intrathecally, intracerebroventricularly, iontophoretically, transmucosally, intravitreally, or intramuscularly. Administration includes self-administration, the administration by another or administration by use of a device (e.g., an infusion pump).

As used herein, to “ameliorate” or “ameliorating” a disease, disorder or condition refers to results that, in a statistical sample or specific subject, make the occurrence of the disease, disorder or condition (or a sign, symptom or condition thereof) better or more tolerable in a sample or subject administered a therapeutic agent relative to a control sample or subject.

As used herein the terms “carrier” or “pharmaceutically acceptable carrier” refer to a diluent, adjuvant, excipient, or vehicle with which a compound/drug product/composition (including a medicament) is administered or formulated for administration. Non-limiting examples of such pharmaceutically acceptable carriers include liquids, such as water, saline, oils and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the phrase “delaying the onset of” refers to, in a statistical sample, postponing, hindering the occurrence of a disease, disorder or condition, or causing one or more signs, symptoms or conditions of a disease, disorder or condition to occur more slowly than normal, in a sample or subject administered a therapeutic agent relative to a control sample or subject.

As used herein, the term “effective amount” refers to a quantity of a compound/composition/drug product sufficient to achieve a desired therapeutic and/or prophylactic effect, e.g., an amount that treats, prevents, inhibits, ameliorates, or delays the onset of the disease, disorder or condition, or the physiological signs, symptoms or conditions of the disease or disorder. In the context of therapeutic or prophylactic applications, in some embodiments, the amount of a compound/composition/drug product administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors. The compounds/compositions/drug products can also be administered in combination with one or more additional therapeutic compounds/agents (a so called “co-administration” where, for example, the additional therapeutic agent could be administered simultaneously, sequentially or by separate administration).

As used herein, “inhibit” or “inhibiting” refers to the reduction in a sign, symptom or condition (e.g. risk factor) associated with a disease, disorder or condition associated with a Huntington's disease and/or a HTT proteinopathy by an objectively measurable amount or degree compared to a control. In one embodiment, inhibit or inhibiting refers to the reduction by at least a statistically significant amount compared to a control (or control subject). In one embodiment, inhibit or inhibiting refers to a reduction by at least 5 percent compared to control (or control subject). In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control (or control subject).

As used herein, “prevention” or “preventing” of a disease, disorder, or condition refers to results that, in a statistical sample, exhibit a reduction in the occurrence of the disease, disorder, or condition in a sample or subject administered a therapeutic agent relative to a control sample or subject, or exhibit a delay in the onset of one or more symptoms of the disease, disorder, or condition relative to the control sample or subject. Such prevention is sometimes referred to as a prophylactic treatment.

As used herein, the term “separate” therapeutic use refers to an administration of at least two active ingredients (e.g. therapeutic agents) at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this definition.

As used herein, the term “simultaneous” therapeutic use refers to the administration of at least two active ingredients by the same route and at the same time or at substantially the same time.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

It is also to be appreciated that the various modes of treatment or prevention of medical conditions as described herein are intended to mean “substantial,” which includes total but also less than total treatment or prevention, and wherein some biologically or medically relevant result is achieved.

As used herein, a “synergistic therapeutic effect” refers to a greater-than-additive therapeutic effect which is produced by a combination of at least two agents, and which exceeds that which would otherwise result from the individual administration of the agents. For example, lower doses of one or more agents may be used in treating Huntington's disease and/or a HTT proteinopathy.

As used herein, the terms “treating” or “treatment” refer to therapeutic treatment, wherein the object is to reduce, alleviate or slow down (lessen) a pre-existing disease or disorder, or its related signs, symptoms or conditions. By way of example, but not by way of limitation, a subject is successfully “treated” for a disease if, after receiving an effective amount of the compound/composition/drug product or a pharmaceutically acceptable salt, stereoisomer, tautomer, hydrate, and/or solvate thereof, the subject shows observable and/or measurable reduction in or absence of one or more signs, symptoms or conditions associated with the disease, disorder or condition. It is also to be appreciated that the various modes of treatment of medical conditions as described are intended to mean “substantial,” which includes total alleviation of conditions, signs or symptoms of the disease or disorder, as well as “partial,” where some biologically or medically relevant result is achieved.

### III. Huntington's Disease

Huntington's disease is a progressive neurodegenerative disorder that causes motor impairment, cognitive decline and behavioral modifications. Huntington's disease is characterized as a proteinopathy, associated with aggregates of huntingtin protein (HTT) in the brain. Though the pathology of the disease involves accumulation of huntingtin protein in the brain, Huntington's disease is a genetic autosomal dominant disorder caused by a mutation in the gene HTT that results in a CAG repeat expansion. The age of onset and aggressiveness of the disease appears to closely correlate with the length of the CAG repeat expansion (Lee et al., Neurology (2012) 78: 690-695). The repeat expansion appears to increase during the lifetime of the subject (Kacher et al. eLife (2021) 10: e64674). The disease often becomes symptomatic between the ages of 30 to 50 but can occur at anytime, even in early childhood (at age 2 or up) or at advanced age (e.g., >70). The average lifespan after diagnosis of Huntington's disease is about 10 to 30 years with an average of about 15-17 years. No effective treatments for Huntington's disease are available.

There are various signs and symptoms of Huntington's disease. Symptoms of Huntington's disease that are associated with motor impairment include weight loss, stumbling, clumsiness, loss of coordination, loss of control of movements, difficulty swallowing, difficulty speaking and eventually loss of ambulation. Symptoms of Huntington's disease that are associated with behavioral modifications include personality changes, loss of initiative, mood changes, apathy, irritability, aggression, anger, depression, and frustration. Symptoms of Huntington's disease that are associated with cognitive decline include difficulty focusing, lapses in short term memory, diminishing organizational skills, disorientation, and loss of cognitive skills.

The only therapeutic agents currently approved by the FDA for Huntington's disease are Xenazine® (tetrabenazine) and Austedo® (deutetrabenazine), but these are only approved for reducing the motor impairment associated with the disease. Some other drugs under evaluation for treating various signs and symptoms of Huntington's disease include Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine). There are no approved therapeutic agents for treating the underlying causes of Huntington's disease itself.

### IV. Peptidomimetics

In some embodiments, the present disclosure provides a peptidomimetic compound of Formula (II), or a pharmaceutically acceptable salt, stereoisomer, tautomer, hydrate, and/or solvate thereof:

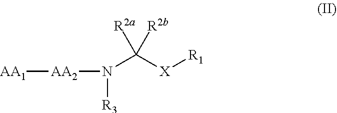

wherein
AA₁ is selected from

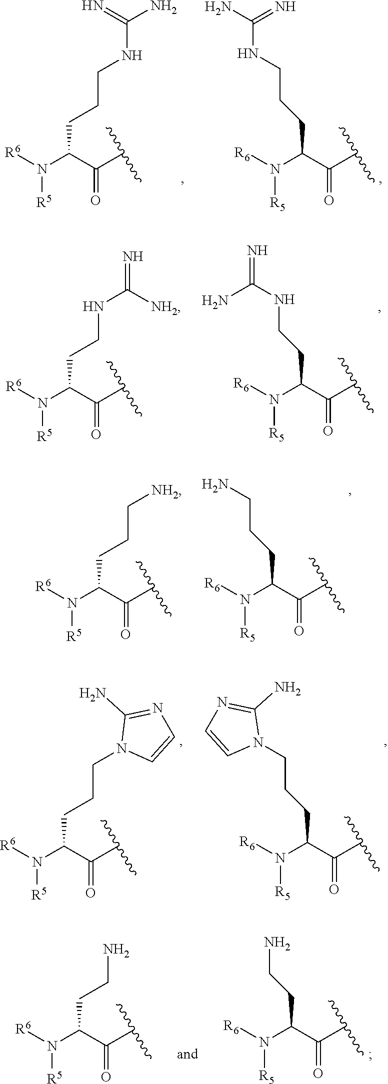

AA₂ is selected from

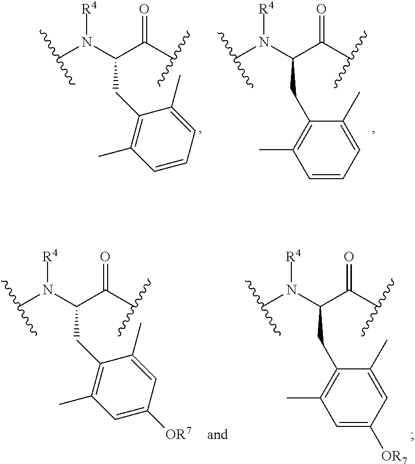

R¹ is selected from

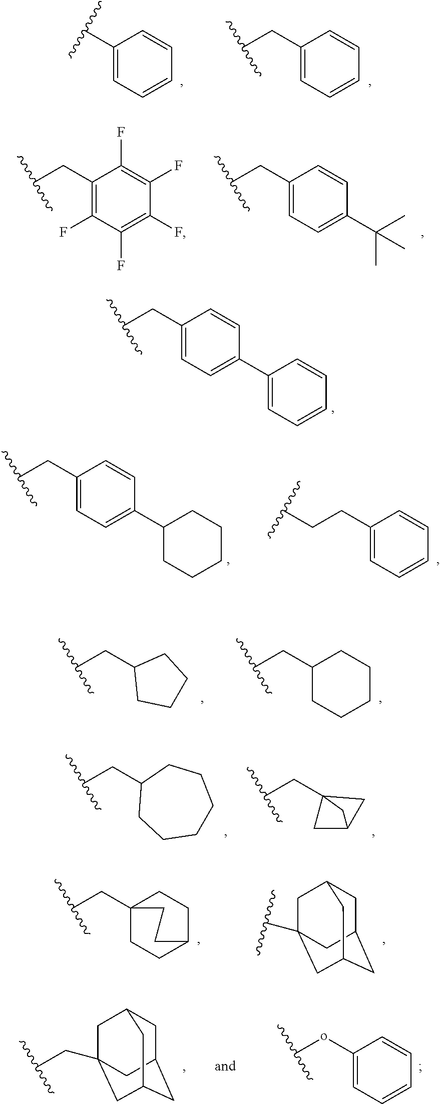

R^{2a} is selected from

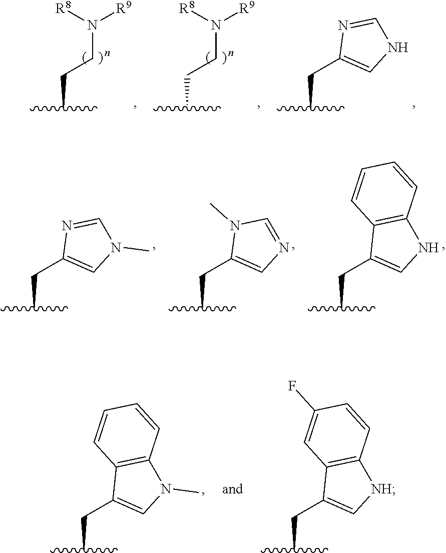

R^{2b} is H or Me;
R³ and R⁴ are independently selected from H and (C₁-C₆)alkyl;
R⁵ and R⁶ are independently H, methyl, ethyl, propyl, cyclopropyl, or cyclobutyl; or R⁵ and R⁶ together with the N atom to which they are attached form a 4-6-membered heterocyclyl;
R⁷ is selected from H, (C₁-C₆)alkyl, cycloalkyl, and aryl; R⁸ and R⁹ are independently selected from H, (C₁-C₆)alkyl, cycloalkyl, and aryl; or R⁸ and R⁹ together with the N atom to which they are attached form a 4-6-membered heterocyclyl;
n is 1, 2, or 3;
X is selected from

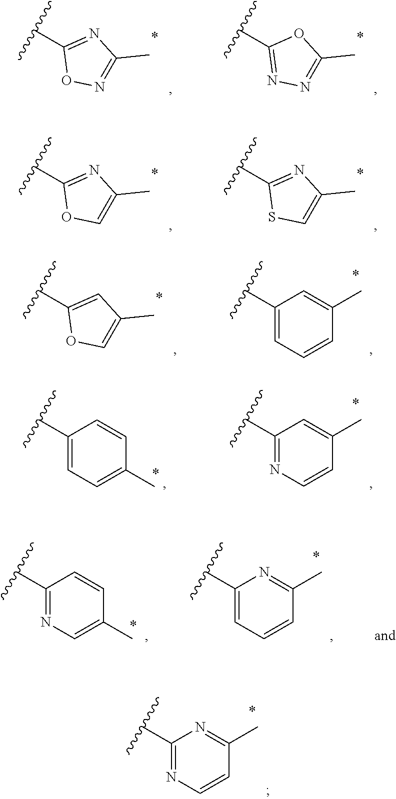

and
* denotes the point of attachment of X to R¹.

In some embodiments, AA₁ is

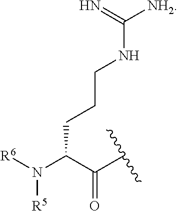

In some embodiments, AA₁ is

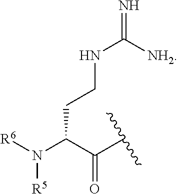

In some embodiments, AA₁ is

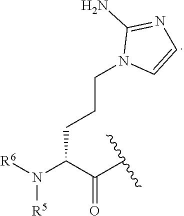

In some embodiments, AA₁ is

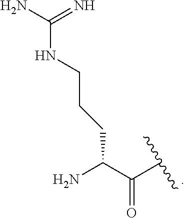 [SMILES: *C(=O)[C@H](N)CCCNC(=N)N]

In some embodiments, AA₁ is

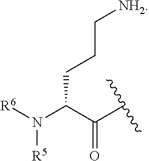

In some embodiments, AA₁ is

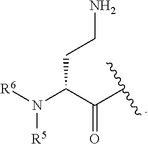

In some embodiments, AA₂ is

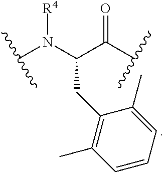

In some embodiments, AA₂ is

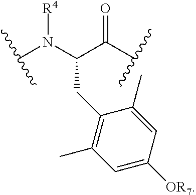

In some embodiments AA₂ is

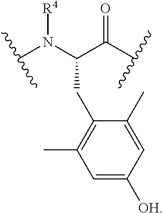

In some embodiments, AA₂ is

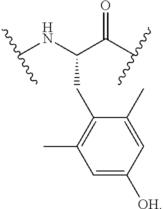 [SMILES: *N[C@@H](Cc1c(C)cc(O)cc1C)C(*)=O]

In some embodiments, R¹ is

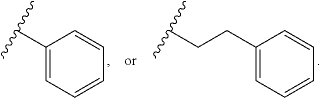 [SMILES: *c1ccccc1 ; *CCc1ccccc1]

In some embodiments, R¹ is

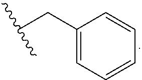 [SMILES: *Cc1ccccc1]

In some embodiments, R¹ is

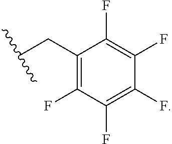 [SMILES: *Cc1c(F)c(F)c(F)c(F)c1F]

In some embodiments, R¹ is

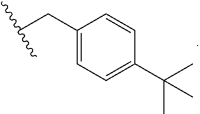 [SMILES: *Cc1ccc(C(C)(C)C)cc1]

In some embodiments, R¹ is

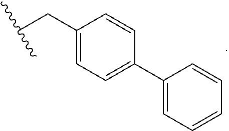 [SMILES: *Cc1ccc(-c2ccccc2)cc1]

In some embodiments, R¹ is

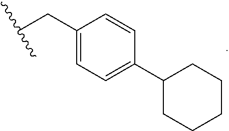 [SMILES: *Cc1ccc(C2CCCCC2)cc1]

In some embodiments R¹ is

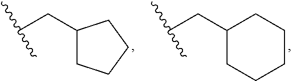

or

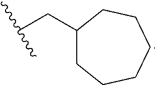 [SMILES: *CC1CCCCCC1]

In some embodiments, R¹ is

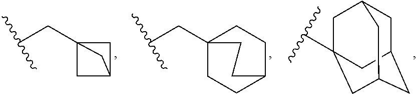

or

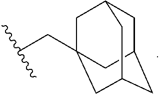 [SMILES: *CC12CC3CC(CC(C3)C1)C2]

In some embodiments, R¹ is

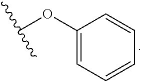 [SMILES: *Oc1ccccc1]

In some embodiments, R^{2a} is

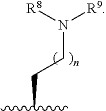

In some embodiments, R^{2a} is

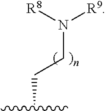

In some embodiments, R^{2a} is

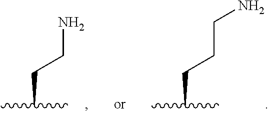

In some embodiments, R^{2a} is

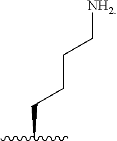

In some embodiments, R^{2a} is

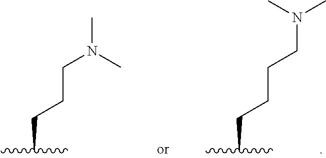

In some embodiments, R^{2a} is

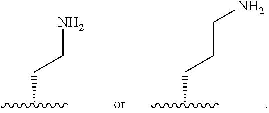

In some embodiments, R^{2a} is

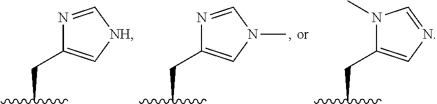

In some embodiments, R^{2a} is

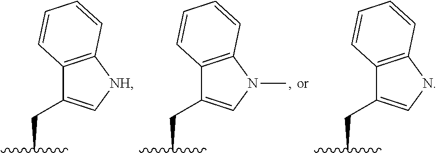

In some embodiments, R^{2b} is H. In some embodiments, R^{2b} is methyl.

In some embodiments, R³ is H. In some embodiments, R³ is (C₁-C₆)alkyl. In some embodiments, R³ is methyl, ethyl, n-propyl, i-propyl, n-butyl, i-butyl, or t-butyl. In some embodiments, R³ is methyl. In some embodiments, R³ is ethyl.

In some embodiments, R⁴ is H. In some embodiments, R⁴ is (C₁-C₆)alkyl. In some embodiments, R⁴ is methyl, ethyl, n-propyl, i-propyl, n-butyl, i-butyl, or t-butyl. In some embodiments, R⁴ is methyl. In some embodiments, R⁴ is ethyl.

In some embodiments, R³ and R⁴ are the same. In some embodiments, R³ and R⁴ are different.

In some embodiments, R⁵ is H. In some embodiments, R⁵ is methyl.

In some embodiments, R⁶ is H. In some embodiments, R⁶ is methyl.

In some embodiments, R⁵ and R⁶ are the same. In some embodiments, R⁵ and R⁶ are different. In some embodiments, both R⁵ and R⁶ are H.

In some embodiments, R⁵ and R⁶ together with the N atom to which they are attached form a 4-6-membered heterocyclyl. In some embodiments, the heterocyclyl is a 4-6 membered ring. In some embodiments, the heterocyclyl is azetidinyl, pyrrolidinyl, or piperidinyl.

In some embodiments, R⁷ is H. In some embodiments, R⁷ is (C₁-C₆)alkyl. In some embodiments, R⁷ is methyl, ethyl, n-propyl, i-propyl, n-butyl, i-butyl, or t-butyl. In some embodiments, R⁷ is methyl. In some embodiments, R⁷ is cycloalkyl. In some embodiments, R⁷ is cyclopropyl, cyclobutyl, cyclopropyl, or cyclohexyl. In some embodiments, R⁷ is aryl. In some embodiments, R⁷ is phenyl.

In some embodiments, R⁸ is H. In some embodiments, R⁸ is (C₁-C₆)alkyl. In some embodiments, R⁸ is methyl, ethyl, n-propyl, i-propyl, n-butyl, i-butyl, or t-butyl. In some embodiments, R⁸ is methyl. In some embodiments, R⁸ is cycloalkyl. In some embodiments, R⁸ is cyclopropyl, cyclobutyl, cyclopropyl, or cyclohexyl. In some embodiments, R⁸ is aryl. In some embodiments, R⁸ is phenyl.

In some embodiments, R⁹ is H. In some embodiments, R⁹ is (C₁-C₆)alkyl. In some embodiments, R⁹ is methyl, ethyl, n-propyl, i-propyl, n-butyl, i-butyl, or t-butyl. In some embodiments, R⁹ is methyl. In some embodiments, R⁹ is cycloalkyl. In some embodiments, R⁹ is cyclopropyl, cyclobutyl, cyclopropyl, or cyclohexyl. In some embodiments, R⁹ is aryl. In some embodiments, R⁹ is phenyl.

In some embodiments, R⁸ and R⁹ are the same. In some embodiments, R⁸ and R⁹ are different. In some embodiments, both R⁸ and R⁹ are H. In some embodiments, R⁸ and R⁹ together with the N atom to which they are attached form a 4-6-membered heterocyclyl. In some embodiments, the heterocyclyl is a 4-6 membered ring. In some embodiments, the heterocyclyl is azetidinyl, pyrrolidinyl, or piperidinyl.

In some embodiments, X is

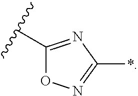

In some embodiments, X is

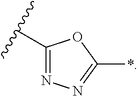

In some embodiments, X is

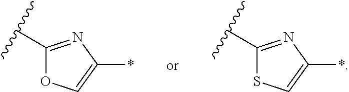

In some embodiments, X is

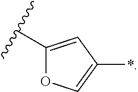

In some embodiments, X is

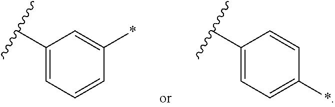

In some embodiments, X is

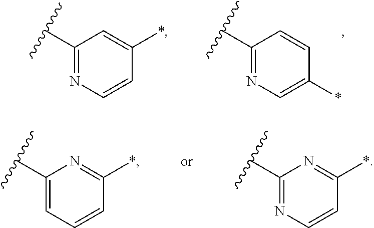

In some embodiments, n is 1. In some embodiments, n is 2. In some embodiments, n is 3.

In some embodiments, the peptidomimetic is a small molecule peptidomimetic. In some embodiments, the peptidomimetic is a peptidomimetic of Formula I or Formula Ia. The peptidomimetics disclosed herein can exist in unsolvated forms as well as solvated forms, including hydrated forms. Solvated forms can exist, for example, because it is difficult or impossible to remove all the solvent from the peptidomimetic post synthesis. In general, the solvated forms are equivalent to unsolvated forms and are encompassed within the scope of the present application. Certain peptidomimetics of the present application may exist in multiple crystalline or amorphous forms. Certain peptidomimetics of the present application may exist in various tautomeric forms. Certain peptidomimetics of the present application may exist in various salt forms. In general, all physical forms are equivalent for the uses contemplated by the present application and are intended to be within the scope of the present application. The chiral centers of the peptidomimetic disclosed herein may be in either the R— or S— configuration as discussed in more detail below.

### V. Chiral/Stereochemistry Considerations

Peptidomimetics described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The peptidomimetics additionally encompasses compounds described herein as individual isomers substantially free of other isomers, and alternatively, as mixtures of various isomers.

As used herein, a pure enantiomeric peptidomimetic is substantially free from other enantiomers or stereoisomers of the compound (i.e., in enantiomeric excess). In other words, an “S” form of the compound is substantially free from the “R” form of the compound and is, thus, in enantiomeric excess of the “R” form. With respect to amino acids (which are more commonly described in terms of “D” and “L” enantiomer, it is to be understood that for a “D”-amino acid the configuration is “R” and for an “L”-amino acid, the configuration is “S”. In some embodiments, ‘substantially free’, refers to: (i) an aliquot of an “R” form compound that contains less than 2% “S” form; or (ii) an aliquot of an “S” form compound that contains less than 2% “R” form. The term “enantiomerically pure” or “pure enantiomer” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition or medicament comprising enantiomerically pure “R” form compound can comprise, for example, about 90% excipient and about 10% enantiomerically pure “R” form compound. In certain embodiments, the enantiomerically pure “R” form compound in such compositions can, for example, comprise, at least about 95% by weight “R” form compound and at most about 5% by weight “S” form compound, by total weight of the compound. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the compound. In certain embodiments, the active ingredient can be formulated with little or no excipient or carrier.

### VI. Synthesis of Peptidomimetics

The peptidomimetic compounds described herein may be prepared, in whole or in part using well known peptide synthesis methods, such as conventional liquid-phase (also known as solution-phase) peptide synthesis or solid-phase peptide synthesis, or by peptide synthesis by means of an automated peptide synthesizer (Kelley et al., Genetics Engineering Principles and Methods, Setlow, J. K. eds., Plenum Press NY. (1990) Vol. 12, pp. 1 to 19; Stewart et al., Solid-Phase Peptide Synthesis (1989) W. H.; Houghten, Proc. Natl. Acad. Sci. USA (1985) 82: p.5132). The peptidomimetic thus produced can be collected or purified by a routine method, for example, chromatography, such as gel filtration chromatography, ion exchange column chromatography, affinity chromatography, reverse phase column chromatography, and HPLC, ammonium sulfate fractionation, ultrafiltration, and immunoadsorption.

In a solid-phase peptide synthesis, peptides are typically synthesized from the carbonyl group side (C-terminus) to amino group side (N-terminus) of the amino acid chain. In certain embodiments, an amino-protected amino acid is covalently bound to a solid support material through the carboxyl group of the amino acid, typically via an ester or amido bond and optionally via a linking group. The amino group may be deprotected and reacted with (i.e., “coupled” with) the carbonyl group of a second amino-protected amino acid using a coupling reagent, yielding a dipeptide bound to a solid support. After coupling, the resin is optionally treated with a capping reagent to thereby cap (render inactive towards subsequent coupling steps) any unreacted amine groups. These steps (i.e., deprotection, coupling and optionally capping) may be repeated to form the desired peptide chain. Once the desired peptide chain is complete, the peptide may be cleaved from the solid support. The peptidomimetics (or peptide portions thereof) can also be prepared in solution.

In certain embodiments, the protecting groups used on the amino groups of the amino acid residues (of peptides and/or peptidomimetics) include 9-fluorenylmethyloxycarbonyl group (Fmoc) and t-butyloxycarbonyl (Boc). The Fmoc group is removed from the amino terminus with base while the Boc group is removed with acid. In alternative embodiments, the amino protecting group may be formyl, acrylyl (Acr), benzoyl (Bz), acetyl (Ac), trifluoroacetyl, substituted or unsubstituted groups of aralkyloxycarbonyl type, such as the benzyloxycarbonyl (Z), p-chlorobenzyloxycarbonyl, p-bromobenzyloxycarbonyl, p-nitrobenzyloxycarbonyl, p-methoxybenzyloxycarbonyl, benzhydryloxycarbonyl, 2(p- biphenylyl)isopropyloxycarbonyl, 2-(3,5-dimethoxyphenyl)isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methylsulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group.

Many amino acids bear reactive functional groups in the side chain. In certain embodiments, such functional groups are protected in order to prevent the functional groups from reacting with the incoming amino acid. The protecting groups used with these functional groups must be stable to the conditions of peptide and/or peptidomimetic synthesis, but may be removed before, after, or concomitantly with cleavage of the peptide from the solid support (if support bound) or upon final deprotection in the case of solution-phase synthesis. Further reference is also made to: Isidro-Llobet, A., Alvarez, M., Albericio, F., “Amino Acid-Protecting Groups”; Chem. Rev., 109: 2455-2504 (2009) as a comprehensive review of protecting groups commonly used in peptide synthesis (which protection groups can also be used in peptidomimetic synthesis where the peptidomimetic comprises functional groups found in peptides).

In certain embodiments, the solid support material used in the solid-phase peptide synthesis method is a gel-type support such as polystyrene, polyacrylamide, or polyethylene glycol. Alternatively, materials such as controlled-pore glass, cellulose fibers, or polystyrene may be functionalized at their surface to provide a solid support for peptide synthesis.

Coupling reagents that may be used in the solid-phase (or solution-phase) peptide synthesis described herein are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC) and its HCl salt (EDC·HCl), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis[[4-(2,2-dimethyl-1,3-dioxolyl)]-methyl]carbodiimide (BDDC), and N,N-dicyclopentylcarbodiimide. DCC is a preferred coupling reagent. Other coupling agents include HATU and HBTU, generally used in combination with an organic base such as DIEA and a hindered pyridine-type base such as lutidine or collidine.

In some embodiments, the amino acids can be activated toward coupling to a peptide or peptidomimetic by forming N-carboxyanhydrides as described in Fuller et al., Urethane-Protected α-Amino Acid N-Carboxyanhydrides and Peptide Synthesis, Biopolymers (Peptide Science), Vol. 40, 183-205 (1996) and WO2018/034901.

Methods for preparation of representative small molecule peptidomimetics such as Compound I and Compound (Ia) can be found WO2019/118878, incorporated herein by reference. More specifically, the synthesis of the Compound (Ia) is specifically described in WO2019/118878 with respect to the synthesis of Compound 7a as described therein. Many other similar peptidomimetics were prepared as described therein—therefore demonstrating the feasibility and versatility of such methodologies to produce many different small molecule peptidomimetics.

### VI. Pharmaceutical Compositions, Routes of Administration, and Dosing

The small molecule peptidomimetics disclosed herein can be used, alone or in combination, with other therapeutically active ingredients to address the needs of subjects suffering from Huntington's disease and/or a HTT proteinopathy. In order to be administered to a subject in need thereof, the small molecule peptidomimetic will generally need to be formulated for the suitable route of administration. The formulated product can be considered a composition or medicament comprising the small molecule peptidomimetic and optionally one or more additional active therapeutic agents.

In certain embodiments, a pharmaceutical composition of the present application may further comprise at least one additional therapeutic agent other than a small molecule peptidomimetic (e.g., an additional therapeutic agent for use in a combination therapy). The at least one additional therapeutic agent can be an agent useful in the treatment of Huntington's disease or a HTT proteinopathy. Thus, in some embodiments, pharmaceutical compositions of the present application can be prepared, for example, by combining one or more compounds of the present application (e.g., a small molecule peptidomimetic) with a pharmaceutically acceptable carrier and, optionally, one or more additional therapeutical agents.

Pharmaceutical compositions of the present application may contain an effective amount of a therapeutic compound/agent (or compounds/agents) as described herein and may optionally be disbursed (e.g., dissolved, suspended or otherwise) in a pharmaceutically acceptable carrier. The components of the pharmaceutical composition(s) may also be capable of being commingled with the compounds of the present application, and with each other, in a manner such that there is no interaction that would substantially impair the desired pharmaceutical efficiency.

As stated above, an “effective amount” refers to any amount of the active compound (or compounds; alone or as formulated) that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic (i.e., preventative) or therapeutic treatment regimen can be planned which does not cause substantial unwanted toxicity and yet is effective to treat the particular condition or disease of a particular subject. The effective amount for any particular indication can vary depending on such factors as the disease, disorder or condition being treated, the particular compound or compounds being administered, the size of the subject, or the severity of the disease, disorder or condition. The effective amount may be determined during pre-clinical trials and/or clinical trials by methods familiar to physicians and clinicians. One of ordinary skill in the art can empirically determine the effective amount of a particular compound and/or other therapeutic agent(s) without necessitating undue experimentation. A maximum dose may be used, that is, the highest safe dose according to some medical judgment.

Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein. A dose may be administered by oneself, by another or by way of a device (e.g., a pump).

For any compound described herein the therapeutically effective amount can, for example, be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Compounds (alone or as formulated in a pharmaceutical composition/medicament) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing, any of the animal model system known in the art can be used prior to administration to human subjects. In some embodiments, dosing can be tested directly in humans.

Dosage, toxicity and therapeutic efficacy of any therapeutic compounds/agents, compositions (e.g., formulations or medicaments), other therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, in such cases it may be prudent to design a delivery system that targets such compounds to the site of affected tissue in order to minimize potential damage to uninfected cells and, thereby, reduce side effects.

In some embodiments, an effective amount of a therapeutic compound/agent disclosed herein sufficient for achieving a therapeutic or prophylactic effect, can range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 100 mg/kg body weight every day, every two days or every three days or within the range of 1-100 mg/kg every week, every two weeks or every three weeks. In some embodiments, a single dosage of a therapeutic compound/agent disclosed herein ranges from 0.001-10,000 micrograms per kg body weight. In some embodiments, a therapeutic compound/agent disclosed herein dissolved or suspended in a carrier range from 0.2 to 2000 micrograms per delivered milliliter. In some embodiments, the dose regimen meets pharmacokinetic target concentrations in target tissues to achieve a desired therapeutic outcome.

An exemplary treatment regime can entail administration once per day, twice per day, thrice per day, once a week, or once a month. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

In some embodiments, a therapeutically effective amount of a therapeutic compound/agent disclosed herein may be defined as a concentration of compound existing at the target tissue of 10⁻¹² to 10⁻⁴ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., oral, systemic, topical, subcutaneous, intra-nasal, parenteral infusion or transdermal application).

In some embodiments, intravenous or subcutaneous administration of a compound (alone or as formulated) may typically be from 0.01 μg/kg/day to 80 mg/kg/day. In some embodiments, intravenous or subcutaneous administration of a compound (alone or as formulated) may typically be from 0.01 μg/kg/day to 100 μg/kg/day. In some embodiments, intravenous or subcutaneous administration of a compound (alone or as formulated) may typically be from 0.1 μg/kg/day to 10 mg/kg/day. In some embodiments, intravenous or subcutaneous administration of a compound (alone or as formulated) may typically be from 10 μg/kg/day to 2 mg/kg/day. In some embodiments, intravenous or subcutaneous administration of a compound (alone or as formulated) may typically be from 500 μg/kg/day to 5 mg/kg/day. In some embodiments, intravenous or subcutaneous administration of a compound (alone or as formulated) may typically be from 1 mg/kg/day to 100 mg/kg/day. In some embodiments, intravenous or subcutaneous administration of a compound (alone or as formulated) may typically be from 1 mg/kg/day to 50 mg/kg/day.

Generally, daily oral doses of a compound (alone or as formulated) will be, for human subjects, from about 0.01 micrograms/kg per day to 250 milligrams/kg per day. In some embodiments, daily oral doses of a compound (alone or as formulated) will be, for human subjects, from about 1 milligrams/kg per day to 100 milligrams/kg per day or from about 10 milligrams/kg per day to 75 milligrams/kg per day or It is expected that oral doses of a compound (alone or as formulated) in the range of 0.1 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For use in therapy, an effective amount of the compound (alone or as formulated) can be administered to a subject by any mode that delivers the compound to the desired surface. Administering a pharmaceutical composition may be accomplished by any means known to the skilled artisan. Routes of administration include but are not limited to oral, topical, intranasal, systemic, intravenous, subcutaneous, intraperitoneal, intradermal, intraocular, ophthalmical, intrathecal, intracerebroventricular, iontophoretical, transmucosal, intravitreal, or intramuscular administration. Administration includes self-administration, the administration by another and administration by a device (e.g., a pump).

A therapeutic compound/agent disclosed herein can be delivered to the subject in a formulation or medicament (i.e., a pharmaceutical composition). Formulations and medicaments can be prepared by, for example, dissolving or suspending a therapeutic compound/agent disclosed herein in water, a pharmaceutically acceptable carrier, salt, (e.g., NaCl or sodium phosphate), buffering agents, preservatives, compatible carriers, adjuvants, and optionally other therapeutically acceptable ingredients.

The pharmaceutical compositions (e.g., a formulation or medicament) can include a carrier (e.g., a pharmaceutically acceptable carrier), which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars (e.g., trehalose), polyalcohols such as mannitol, sorbitol, or sodium chloride in the composition. Prolonged absorption of the injectable compositions can be brought about by including in the composition an agent that delays absorption, for example, aluminum monostearate or gelatin.

Solutions or suspensions (e.g., a formulation or medicament) used for parenteral, intradermal, subcutaneous or intraocular application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 1, 2, 3, 4, 5, 6, 7 days or more of treatment).

The therapeutic compounds/agents or pharmaceutical compositions, when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Systemic formulations include those designed for administration by injection, e.g., subcutaneous, intravenous, intramuscular, intrathecal or intraperitoneal injection, as well as those designed for transdermal, transmucosal oral or pulmonary administration.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g., a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and may be preserved against the contaminating action of microorganisms such as bacteria and fungi.

Sterile injectable solutions (e.g., a formulation or medicament) can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

For oral administration, the compounds can be formulated readily by combining the active compound(s) with pharmaceutically acceptable carriers well known in the art. Such carriers enable the compounds of the present application to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above that may be chemically modified so that oral delivery of the derivative is efficacious. Generally, the chemical modification contemplated is the attachment of at least one moiety to the therapeutic agent(s), ingredient(s), and/or excipient(s), where said moiety permits (a) inhibition of acid hydrolysis; and (b) uptake into the blood stream from the stomach or intestine. Also desired is the increase in overall stability of the therapeutic agent(s), ingredient(s), and/or excipient(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of the compound of the present application (or derivative) or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic compound/agent or pharmaceutical composition can be included in the formulation as fine multi-particulates in the form of granules or pellets of particle size about 1-2 mm. The formulation of the material for capsule administration could also be as a powder, lightly compressed plugs or even as tablets. The therapeutic compound/agent or pharmaceutical composition could be prepared by compression.

Colorants and flavoring agents may all be included. For example, the compound or pharmaceutical composition of the present application (or derivative) may be formulated and then further contained within an edible product, such as a refrigerated beverage containing colorants and flavoring agents.

One may dilute or increase the volume of the therapeutic compound/agent or pharmaceutical composition with an inert material. These diluents could include carbohydrates, especially mannitol, lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic compound/agent or composition into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic compound/agent or composition (e.g. medicament) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the present application or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Solutions, gels, ointments, creams or suspensions may be administered topically. The compounds may also be formulated in rectal or vaginal compositions such as suppositories or retention enemas, e.g., containing conventional suppository bases such as cocoa butter or other glycerides.

For administration by inhalation, compounds or compositions (e.g. medicament) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament and/or other therapeutic compound/agent can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic compound/agent and a suitable powder base such as lactose or starch. Alternatively, the active compounds may be in powder form for constitution with a suitable vehicle, e.g., sterile pyrogen-free water, before use.

Nasal delivery of a therapeutic compound/agent or pharmaceutical composition of the present application is also contemplated. Nasal delivery allows the passage of a therapeutic compound/agent or pharmaceutical composition to the blood stream directly after administering the therapeutic compound/agent or pharmaceutical composition to the nose, without the necessity for deposition of the product in the lung. Formulations for nasal delivery include those with dextran or cyclodextran.

For nasal administration, a useful device is a small, hard bottle to which a metered dose sprayer is attached. In some embodiments, the metered dose is delivered by drawing the pharmaceutical composition of the present application solution into a chamber of defined volume, which chamber has an aperture dimensioned to aerosolize and aerosol formulation by forming a spray when a liquid in the chamber is compressed. The chamber is compressed to administer the therapeutic compound/agent or pharmaceutical composition. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic compound/agent or pharmaceutical composition.

Also contemplated herein is pulmonary delivery of the compounds disclosed herein. The compound or pharmaceutical composition is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this technology are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this technology are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of the compound(s)/therapeutic agent(s). Typically, each formulation is specific to the type of device employed and may involve the use of an appropriate propellant material, in addition to the usual diluents, adjuvants and/or carriers useful in therapy. Also, the use of liposomes, microcapsules, microspheres, nanoparticles, nanospheres, inclusion complexes, or other types of carriers is contemplated. Chemically modified compound of the present application may also be prepared in different formulations depending on the type of chemical modification or the type of device employed.

Formulations suitable for use with a nebulizer, either jet or ultrasonic, can, for example, comprise a compound/therapeutic agent of the present application (or derivative) dissolved in water at a concentration of about 0.01 to 50 mg of biologically active compound per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the present application caused by atomization of the solution in forming the aerosol.

Formulations for use with a metered-dose inhaler device may generally comprise a finely divided powder containing the compound of the present application (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device may comprise a finely divided dry powder containing compound of the present application (or derivative) and may also include a bulking agent, such as lactose, sorbitol, sucrose, or mannitol in amounts which facilitate dispersal of the powder from the device, e.g., 50 to 90% by weight of the formulation. The compound(s)/therapeutic agent(s) of the present application (or derivative) can advantageously be prepared in particulate or nanoparticulate form with an average particle size of less than 10 micrometers (μm), most preferably 0.5 to 5 μm, for most effective delivery to the deep lung.

For ophthalmic or intraocular indications, any suitable mode of delivering the therapeutic compounds/agents or pharmaceutical compositions to the eye or regions near the eye can be used. For ophthalmic formulations generally, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983). Nonlimiting examples of pharmaceutical compositions suitable for administration in or near the eye include, but are not limited to, ocular inserts, minitablets, and topical formulations such as eye drops, ointments, and in situ gels. In one embodiment, a contact lens is coated with a pharmaceutical composition comprising a therapeutic compound/agent disclosed herein. In some embodiments, a single dose can comprise from between 0.1 ng to 5000 μg, 1 ng to 500 μg, or 10 ng to 100 μg of the therapeutic compounds/agents or pharmaceutical compositions administered to the eye.

Eye drops can comprise a sterile liquid formulation that can be administered directly to the eye. In some embodiments, eye drops comprise at least one therapeutic compound/agent disclosed herein and may further comprise one or more preservatives. In some embodiments, the optimum pH for eye drops equals that of tear fluid and is about 7.4. For eye drops, the therapeutic compound/agent can be present in the drop solution from about 0.1% to about 5% (w/v or v/v depending on the physical nature (i.e. solid or liquid) of the active ingredient). In some embodiments, the therapeutic compound/agent can be present in the drop solution from about 1% to about 3% (w/v or v/v, as appropriate).

In situ gels are viscous liquids, showing the ability to undergo sol-to-gel transitions when influenced by external factors, such as appropriate pH, temperature, and the presence of electrolytes. This property causes slowing of drug drainage from the eyeball surface and increase of the active ingredient bioavailability. Polymers commonly used in in situ gel formulations include, but are not limited to, gellan gum, poloxamer, silicone containing formulations, silica-based formulations and cellulose acetate phthalate. In some embodiments, the therapeutic compound/agent is formulated into an in-situ gel (as the pharmaceutical composition/medicament).

For topical ophthalmic administration, therapeutic compound/agent or pharmaceutical composition may be formulated as solutions, gels, ointments, creams, suspensions, etc. as are well-known in the art. Ointments are semisolid dosage forms for external use such as topical use for the eye or skin. In some embodiments, ointments comprise a solid or semisolid hydrocarbon base of melting or softening point close to human core temperature. In some embodiments, an ointment applied to the eye decomposes into small drops, which stay for a longer time period in conjunctival sac, thus increasing bioavailability.

Ocular inserts are solid or semisolid dosage forms without disadvantages of traditional ophthalmic drug forms. They are less susceptible to defense mechanisms like outflow through nasolacrimal duct, show the ability to stay in conjunctival sac for a longer period, and are more stable than conventional dosage forms. They also offer advantages such as accurate dosing of one or more therapeutic compounds/agents, slow release of one or more therapeutic compounds/agents with constant speed and limiting of one or more therapeutic compounds'/agents' systemic absorption. In some embodiments, an ocular insert comprises one or more therapeutic compounds/agents as disclosed herein and one or more polymeric materials. The polymeric materials can include, but are not limited to, methylcellulose and its derivatives (e.g., hydroxypropyl methylcellulose (HPMC)), ethylcellulose, polyvinylpyrrolidone (PVP K-90), polyvinyl alcohol, chitosan, carboxymethyl chitosan, gelatin, and various mixtures of the aforementioned polymers. An ocular insert can comprise silica. An ocular insert can comprise liposomes, nanoparticles or microparticles of degradable or biodegradable polymer (as described in more detail below).

Minitablets are biodegradable, solid drug forms, that transit into gels after application to the conjunctival sac, thereby extending the period of contact between active ingredient (i.e. the therapeutic compounds/agents disclosed herein) and the eyeball surface, which in turn increases a therapeutic compounds'/agents' bioavailability. The advantages of minitablets include easy application to conjunctival sac, resistance to defense mechanisms like tearing or outflow through nasolacrimal duct, longer contact with the cornea caused by presence of mucoadhesive polymers, and gradual release of the active ingredient from the formulation in the place of application due to the swelling of the outer carrier layers. Minitablets can comprise one or more of the therapeutic compounds/agents disclosed herein and one or more polymers. Nonlimiting examples of polymers suitable for use in in a minitablet formulation include cellulose derivatives, like hydroxypropyl methylcellulose (HPMC), hydroxyethyl cellulose (HEC), sodium carboxymethyl cellulose, ethyl cellulose, acrylates (e.g., polyacrylic acid and its cross-linked forms), Carbopol® or carbomer, chitosan, and starch (e.g., drum-dried waxy maize starch). In some embodiments, minitablets further comprise one or more excipients. Nonlimiting examples of excipients include mannitol and magnesium stearate.

The ophthalmic or intraocular formulations and medicaments may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

In some embodiments, the viscosity of the ocular formulation comprising one or more therapeutic compounds/agents is increased to improve contact with the cornea and bioavailability in the eye. Viscosity can be increased by the addition of hydrophilic polymers of high molecular weight which do not diffuse through biological membranes and which form three-dimensional networks in the water. Nonlimiting examples of such polymers include polyvinyl alcohol, poloxamers, hyaluronic acid, carbomers, and polysaccharides, cellulose derivatives, gellan gum, and xanthan gum.

In addition to the formulations described above, a therapeutic compound/agent disclosed herein may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

In some embodiments, the therapeutic agent(s) is/are administered as a depot formulation wherein the active therapeutic agent(s) is/are encapsulated by, or disposed within, silica-based microparticles. In some embodiments, the ocular formulation can be injected into the eye, for example as a sol-gel (e.g., a silica sol-gel). In some embodiments, the ocular formulation is a depot formulation such as a controlled release formulation (see below). Such controlled release formulation may comprise particles, such as microparticles or nanoparticles.

The pharmaceutical compositions also may comprise suitable solid or gel-phase carriers or excipients. Examples of such carriers or excipients include but are not limited to calcium carbonate, calcium phosphate, various sugars, starches, cellulose derivatives, gelatin, silica/silicone and polymers such as polyethylene glycols.

Suitable liquid or solid pharmaceutical preparation forms can, for example, be aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions can be suitable for use in a variety of drug delivery systems. For a brief review of methods for drug delivery, see Langer R, Science 249:1527-33 (1990).

The therapeutic agent(s), including specifically but not limited to a therapeutic compound/agent disclosed herein, may be provided in particles. Particles as used herein means nanoparticles or microparticles (or in some instances larger particles) which can consist in whole or in part of the therapeutic compound/agent or the other therapeutic agent(s) as described herein. The particles may contain the therapeutic compound(s)/agent(s) in a core surrounded by a coating, including, but not limited to, an enteric coating. The therapeutic compound(s)/agent(s) also may be dispersed throughout the particles. The therapeutic compound(s)/agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic compound(s)/agent(s), any of those materials routinely used in the art of pharmacy and medicine, including, but not limited to, erodible, non-erodible, biodegradable, or nonbiodegradable material or combinations thereof. The particles may be microcapsules which contain the therapeutic compound(s)/agent(s) in a solution or in a semi-solid state. The particles may be of virtually any shape.

Both non-biodegradable and biodegradable polymeric materials can be used in the manufacture of particles for delivering the therapeutic compound(s)/agent(s). Such polymers may be natural or synthetic polymers. The polymer is selected based on the period of time over which release is desired. Bioadhesive polymers of particular interest include bioerodible hydrogels described in Sawhney H S et al. (1993)Macromolecules 26:581-7, the teachings of which are incorporated herein. These include polyhyaluronic acids, casein, gelatin, glutin, polyanhydrides, polyacrylic acid, alginate, chitosan, polyethylene glycols (PEGs), polyvinylalcohols (PVAs), poly(methyl methacrylates), poly(ethyl methacrylates), poly(butylmethacrylate), poly(isobutyl methacrylate), poly-lactic acid (PLA), poly(lactic -co-glycolic) acid (PLGA), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and poly(8-caprolactone) or mixtures of two or more of the foregoing.

Therapeutic compounds/agents or other therapeutic agent(s) or mixtures thereof can be formulated in a carrier system. The carrier can be a colloidal system. The carrier or colloidal system can be a liposome, a phospholipid bilayer vehicle. In one embodiment, therapeutic compound(s)/agent(s) or other therapeutic agent(s) or mixtures thereof can be encapsulated in a liposome while maintaining integrity of the therapeutic compound(s)/agent(s) or other therapeutic agent(s) or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound or other therapeutic agent or mixtures thereof can be embedded in the polymer matrix, while maintaining integrity of the composition. The polymer can be a microparticle or nanoparticle that encapsulates the therapeutic agent or agents. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In some embodiments, the polymer is poly-lactic acid (PLA), poly lactic/glycolic acid (PLGA) or a mixture thereof. The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the nanoparticles or microparticles can be silica-based or silane-based (See for example: WO2002/080977 entitled: “Biodegradable carrier and method for preparation thereof”).

In some embodiments, the therapeutic compound(s)/agent(s) or other therapeutic agent(s) or mixtures thereof are prepared with carriers that will protect the therapeutic compound(s)/agent(s) or other therapeutic agent(s) or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compound(s)/agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom to thereby make it available to the subject. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g. a fibrous mesh constructed from several individual pieces of mesh material) or a plurality of individual compositions where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient(s) for at least 2 days. In some embodiments, the implant or depot formulation is constructed and arranged to deliver therapeutic or prophylactic levels of the active ingredient(s) for at least 7 days. In some embodiments, the implant or depot formulation is constructed and arranged to deliver therapeutic or prophylactic levels of the active ingredient(s) for at least 14 days. In some embodiments, the implant or depot formulation is constructed and arranged to deliver therapeutic or prophylactic levels of the active ingredient(s) for at least 30 days. In some embodiments, the implant or depot formulation is constructed and arranged to deliver therapeutic or prophylactic levels of the active ingredient(s) for at least 60 days. In some embodiments, the implant or depot formulation is constructed and arranged to deliver therapeutic or prophylactic levels of the active ingredient for at least 90 days. In some embodiments, the implant or depot formulation is constructed and arranged to deliver therapeutic or prophylactic levels of the active ingredient(s) for at least 180 days. In some embodiments, the implant or depot formulation is constructed and arranged to deliver therapeutic or prophylactic levels of the active ingredient(s) for at least one year. In some embodiments, the implant or depot formulation is constructed and arranged to deliver therapeutic or prophylactic levels of the active ingredient(s) for 15-30 days. In some embodiments, the implant or depot formulation is constructed and arranged to deliver therapeutic or prophylactic levels of the active ingredient(s) for 30-60 days. In some embodiments, the implant or depot formulation is constructed and arranged to deliver therapeutic or prophylactic levels of the active ingredient(s) for 60-90 days. In some embodiments, the implant or depot formulation is constructed and arranged to deliver therapeutic or prophylactic levels of the active ingredient(s) for 90-120 days. In some embodiments, the implant or depot formulation is constructed and arranged to deliver therapeutic or prophylactic levels of the active ingredient(s) for 120-180 days. In some embodiments, the implant or depot formulation is constructed and arranged to deliver therapeutic or prophylactic levels of the active ingredient(s) for up to one year. In some embodiments, the long-term sustained release implants or depot formulation are well-known to those of ordinary skill in the art and include some of the release systems described above. In some embodiments, such implants or depot formulation can be administered surgically. In some embodiments, such implants or depot formulation can be administered topically or by injection.

### VIII. Formulations and Medicaments

The small molecule peptidomimetics disclosed herein (for example compounds of Formulas I and Ia) can be used, alone or in combination, with other therapeutically active ingredients to address the needs of subjects suffering from Huntington's disease and/or a HTT proteinopathy. In order to be administered to a subject in need thereof, the small molecule peptidomimetic will generally need to be formulated for the suitable route of administration. The formulated product can be considered a composition or medicament comprising the small molecule peptidomimetic and optionally one or more additional active therapeutic agents. For example, if the small molecule peptidomimetic (alone or in combination with another active ingredient) is to be administered to the subject by injection, it will typically be formulated into an injectable liquid or liquid suspension. For example, this could be accomplished by dissolving or suspending the small molecule peptidomimetic in a suitable diluent, adjuvant, excipient, vehicle or pharmaceutically acceptable carrier as described previously herein (See the section above entitled: Pharmaceutical Compositions, Routes of Administration, and Dosing), optionally with one or more optionally one or more additional active therapeutic agents. In some embodiments, the diluent, adjuvant, excipient, vehicle or pharmaceutically acceptable carrier can be water, saline or a buffered aqueous solution.

Similarly, if the small molecule peptidomimetic (alone or in combination with another active therapeutic agents) is to be administered to the subject in oral form, the selected active ingredient(s) can be formulated into a pill, tablet, capsule or other vehicle for such administration as discussed above in the section entitled: “Pharmaceutical Compositions, Routes of Administration, and Dosing” or as otherwise known to those of ordinary skill in the art.

Similarly, the small molecule peptidomimetic (alone or in combination with another active therapeutic agents) can be formulated for ocular administration, buccal administration, topical administration, nasal administration or any other of the modes of administration previously discussed herein or that are known to those of ordinary skill in the art.

In brief, any of the formulations (which can also be referred to as a medicament or composition when formulated for administration to a subject having a certain affliction or medical condition that requires medical attention) described in the section above entitled: “Pharmaceutical Compositions, Routes of Administration, and Dosing” can be applied to produce a composition (i.e. a formulation or medicament) suitable for administration to a subject in need thereof. Thus, in some embodiments, this application is directed to compositions, formulations and medicaments suitable for administration to a subject suffering from, or believed to be suffering from Huntington's disease and/or a HTT proteinopathy.

Thus, in some embodiments, the present disclosure present disclosure provides a formulation or medicament comprising a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof, for use in treating, preventing, inhibiting, ameliorating and/or delaying the onset of Huntington's disease and/or a HTT proteinopathy in a subject in need thereof. In some embodiments, the peptidomimetic is a peptidomimetic of Formula II, or a pharmaceutically acceptable salt, stereoisomer, tautomer, hydrate, and/or solvate thereof. In some embodiments, the peptidomimetic is a peptidomimetic of Formula I or Ia, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, the subject has been diagnosed as having Huntington's disease. In some embodiments, the subject has been diagnosed as having a HTT proteinopathy.

In some embodiments, the formulation or medicament is administered to the subject daily for 2 weeks or more, for 24 weeks or more, for 36 weeks or more, for 48 weeks or more, or for 52 weeks or more. In some embodiments, the formulation or medicament is administered daily for the remainder of the life of the subject.

In some embodiments, the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

In some embodiments, the subject is a mammal. In some embodiments, the mammalian subject is a human.

In some embodiments, the formulation or medicament is administered orally. In some embodiments, the formulation or medicament is administered subcutaneously. In some embodiments, the formulation or medicament is administered topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the formulation or medicament embodies a combination therapy. Thus, in some embodiments, the use of the formulation or medicament further comprises separately, sequentially, or simultaneously administering an additional treatment to the subject. In some embodiments, the additional treatment comprises administration of a therapeutic agent. The additional therapeutic agent can be one used to treat the disease itself or otherwise be used to address symptoms or conditions associated with the disease (in this case Huntington's disease and/or a HTT proteinopathy. In some embodiments, the therapeutic agent is selected from the group consisting of: Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine). In some embodiments, the therapeutic agent is elamipretide (also known as SS-31 or bendavia). In some embodiments, the combination of formulation or medicament and the additional treatment has a synergistic effect in the prevention or treatment of Huntington's disease and/or a HTT proteinopathy.

In some embodiments, the pharmaceutically acceptable salt comprises a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri- (tris-) acid salt). In some embodiments, pharmaceutically acceptable salt comprises a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

### IX. Therapeutic Methods And Related Uses

In one aspect, the present disclosure provides a method for treating, preventing, inhibiting, ameliorating or delaying the onset of Huntington's disease and/or a HTT proteinopathy in a subject in need thereof, comprising administering to the subject a therapeutically effective amount of a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof. In some embodiments, the peptidomimetic is a peptidomimetic of Formula II, or a pharmaceutically acceptable salt, stereoisomer, tautomer, hydrate, and/or solvate thereof. In some embodiments, the peptidomimetic is a peptidomimetic of Formula I or Ia, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, the subject has been diagnosed as having Huntington's disease. In some embodiments, the subject has been diagnosed as having a HTT proteinopathy.

In some embodiments, the peptidomimetic is administered to the subject daily for 2 weeks or more, for 12 weeks or more, for 24 weeks or more, for 36 weeks or more, for 48 weeks or more, or for 52 weeks or more. In some embodiments, once diagnosed, the peptidomimetic is administered daily for the remainder of the life of the subject.

In some embodiments, the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

In some embodiments, the subject is a mammal. In some embodiments, the mammalian subject is a human.

In some embodiments, the peptidomimetic is administered orally. In some embodiments, the peptidomimetic is administered subcutaneously. In some embodiments, the peptidomimetic is administered topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the method involves a combination therapy. In practice of a combination therapy, the method further comprises separately, sequentially, or simultaneously administering an additional treatment to the subject. In some embodiments, the additional treatment comprises administration of a therapeutic agent. In some embodiments, the therapeutic agent is selected from the group consisting of: Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine). In some embodiments, the therapeutic agent is elamipretide (also known as SS-31 or bendavia). In some embodiments, the combination of peptidomimetic and an additional therapeutic treatment has a synergistic effect in the prevention or treatment of Huntington's disease and/or a HTT proteinopathy.

In some embodiments, the pharmaceutically acceptable salt of the peptidomimetic comprises a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri- (tris-) acid salt). In some embodiments, pharmaceutically acceptable salt comprises a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

In one aspect, the present disclosure provides a use of a composition in the preparation of a medicament for treating, preventing, inhibiting, ameliorating or delaying the onset of Huntington's disease and/or a HTT proteinopathy in a subject in need thereof, wherein the composition comprises a therapeutically effective amount of a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof. In some embodiments, the peptidomimetic is a peptidomimetic of Formula II, or a pharmaceutically acceptable salt, stereoisomer, tautomer, hydrate, and/or solvate thereof. In some embodiments, the peptidomimetic is a peptidomimetic of Formula I or Ia, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, the subject has been diagnosed as having Huntington's disease. In some embodiments, the subject has been diagnosed as having a HTT proteinopathy.

In some embodiments, the medicament is administered to the subject daily for 2 weeks or more, for 12 weeks or more, for 24 weeks or more, for 36 weeks or more, for 48 weeks or more, or for 52 weeks or more. In some embodiments, the medicament is administered daily for the remainder of the life of the subject.

In some embodiments, the use of the medicament for the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

In some embodiments, the subject is a mammal. In some embodiments, the mammalian subject is a human.

In some embodiments, the medicament is formulated for oral administration. In some embodiments, the medicament is formulated for subcutaneous administration. In some embodiments, the medicament is formulated for administration, topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the medicament is separately, sequentially, or simultaneously used with an additional treatment. In some embodiments, the additional treatment comprises use of a therapeutic agent. In some embodiments, the therapeutic agent is selected from the group consisting of. Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine). In some embodiments, the therapeutic agent is elamipretide (also known as SS-31 or bendavia). In some embodiments, the combination of medicament and an additional treatment has a synergistic effect in the prevention or treatment of Huntington's disease and/or a HTT proteinopathy.

In some embodiments, the pharmaceutically acceptable salt comprises a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri- (tris-) acid salt). In some embodiments, pharmaceutically acceptable salt comprises a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

In still another aspect, the present disclosure provides a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof, for use in treating, preventing, inhibiting, ameliorating and/or delaying the onset of Huntington's disease and/or a HTT proteinopathy in a subject in need thereof. In some embodiments, the peptidomimetic is a peptidomimetic of Formula II, or a pharmaceutically acceptable salt, stereoisomer, tautomer, hydrate, and/or solvate thereof. In some embodiments, the peptidomimetic is a peptidomimetic of Formula I or Ia, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, the subject has been diagnosed as having Huntington's disease. In some embodiments, the subject has been diagnosed as having a HTT proteinopathy.

In some embodiments, the peptidomimetic is administered to the subject daily for 2 weeks or more, for 24 weeks or more, for 36 weeks or more, for 48 weeks or more, or for 52 weeks or more. In some embodiments, the peptidomimetic is administered daily for the remainder of the life of the subject.

In some embodiments, the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

In some embodiments, the subject is a mammal. In some embodiments, the mammalian subject is a human.

In some embodiments, the peptidomimetic is formulated for administration orally. In some embodiments, the peptidomimetic is formulated for administration subcutaneously. In some embodiments, the peptidomimetic is formulated for administration topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the peptidomimetic is separately, sequentially, or simultaneously used with an additional treatment. In some embodiments, the additional treatment comprises use of a therapeutic agent. In some embodiments, the therapeutic agent is selected from the group consisting of. Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine). In some embodiments, the therapeutic agent is elamipretide (also known as SS-31 or bendavia). In some embodiments, the combination of medicament and an additional treatment has a synergistic effect in the prevention or treatment of Huntington's disease and/or a HTT proteinopathy.

In some embodiments, the pharmaceutically acceptable salt comprises a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri- (tris-) acid salt). In some embodiments, pharmaceutically acceptable salt comprises a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

### EXAMPLES

The present technology is further illustrated by the following examples, which should not be construed as limiting in any way.

### Example 1—Effects of (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (tris-HCl salt (Compound (Ia)) on Motor Deficits and Brain and Peripheral Biomarkers of Disease Progression in R6/2 Mice of Huntington's Disease

Purpose—The objective of this study was to investigate the effects of (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide as its tris-HCl salt (i.e. Compound (Ia)) on motor deficits and biomarkers of brain and peripheral disease progression in transgenic R6/2 mice of Huntington's disease.

Study Summary—A total of 46 male R6/2 mice and 15 male wild-type littermate control mice (WT) were used in the study. The mice were genotyped and the R6/2 mice were divided into different treatment groups based on their pre-treatment body weight. The treatment with Compound (Ia) (0.5 or 5 mg/kg; 10 ml/kg, intraperitoneally (i.p.), QD) or Vehicle (10 ml/kg, i.p., QD) was started at 4 weeks of age. Body weights were measured before the treatment onset and continued twice a week until the end of the study. Fine motor kinematic analysis and rotarod test were performed at 10 weeks of age and the open field activity and clasping behavior were measured at 12 weeks of age for all mice. At 8 and 12 weeks of age the glucose uptake was measured by ¹⁸F-FDG PET and the striatal metabolites were measured using MR spectroscopy for 9-10 mice per group. At 13 weeks of age 9-10 mice per group were subjected to tissue sample collection, and the brain and blood samples were collected. The blood samples were used to prepare peripheral blood mononuclear cells (PBMCs) on which the flow cytometry/luminescence analysis was performed for following assays: mitochondrial reactive oxygen species (ROS) (MitoSox), and ATP levels (Cell TiterGlow).

Altogether 15 R6/2 mice died before the scheduled end point. For those mice that died before the 12-week imaging, new replacement mice were included, starting the dosing at the same age as for the mice initially included in the study.

### Experimental Procedures & Assays

Animals—All animal experiments were performed as specified in the license authorized by the national Animal Experiment Board of Finland (license number: ESAVI/5071/04.10.07/2017) and according to the National Institutes of Health (Bethesda, MD, USA) guidelines for the care and use of laboratory animals. Animals were housed at a standard temperature (21±1.5° C.) and in a light-controlled environment (lights on from 7 am to 8 pm) with ad libitum access to food and water.
Species: Mice
Strain: R6/2 (Stock No: 006494)
Source: Jackson Laboratories (Bar Harbor, ME, USA)
Number of Animals Assigned to Study: 45 R6/2 and 15 WT male mice
Target Age/weight at the Start of In-life Phase: 4 weeks

Each animal was permanently identified with unique permanent identification numbers. Animals were identified using ear marks. After receipt at the Testing Facility, the animals were acclimated at least for seven (7) days prior to the beginning of the in-life phase.

Animals housing—different genotypes were not mixed in the same cage, animals were group housed.

Cages—Individually ventilated caging systems (IVC) and polycarbonate Type II Long cages (Allentown Inc.).

Bedding material—Corn-o'Cobs (Andersons Ltd) and aspen chips (Tapvei Ltd).

Animal enrichment—Environmental enrichment was provided for the animals according to the Government Decree on the Protection of Animals Used for Scientific or Educational Purposes (564/2013). A shelter was provided to all animals: plastic igloos, gnawing material (aspen sticks) and nesting material.

Chow—Standard chow: Teklad Global 2016, Global 2016 pellet Ad libitum.

Water—Tap water. Ad libitum.

Room temperature—21.5±1.5° C.

Veterinary care was available throughout the course of the study and animals were examined by the responsible trained personnel and supervised by veterinarian as warranted by clinical signs or other changes.

Animal Groupings—Animals were grouped as follows:
Group 1: 15 WT mice treated with Vehicle (10 mL/kg, i.p., QD) from 4 to 13 weeks of age
Group 2: 16 R6/2 mice treated with Vehicle (10 mL/kg, i.p., QD) from 4 to 13 weeks of age
Group 3: 15 R6/2 mice treated with Compound (I(a)) (0.5 mg/kg, i.p., QD) from 4 to 13 weeks of age
Group 4: 15 R6/2 mice treated with Compound (I(a)) (5 mg/kg, i.p., QD) from 4 to 13 weeks of age

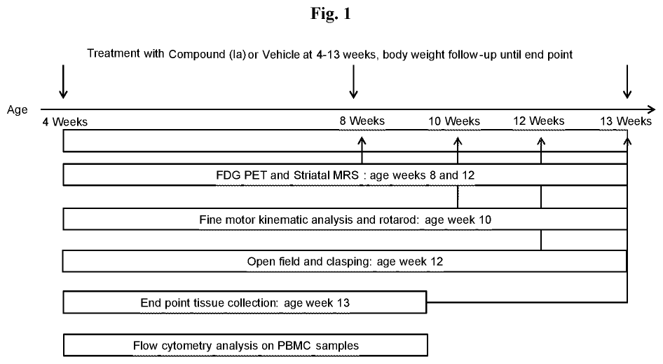

Experimental Design—See FIG. 1; The in-vivo procedures, observations, and measurements listed below were performed for all animals by investigator blinded to the treatment. R6/2 Mice model human Huntington's disease (HD) by expressing a portion of the human HD gene under human gene promoter elements (1 kb of 5 UTR sequence and exon 1 together with ˜120 CAG repeats). Expression of this amino-terminal fragment of the huntingtin protein with its polyglutamine expansion is sufficient to produce the phenotype of human HD.

Test Articles—Dosing formulations were prepared weekly by dissolving Compound (Ia) in sterile saline to the appropriate concentration for administration to the animals and stored at 2-8° C. for seven days once prepared. Unused formulations were discarded at the end of the 7 day period and new dosing formulations were prepared as needed to conduct the study.

Drug delivery—Test article administration was performed by investigators blinded to the treatment. The dose volume for each animal was based on the body weight. The administrations were given i.p., 10 mL/kg. The daily dosing was started at approximately 11 a.m.-1 p.m.

Plasma Bile Acid Level Measurement—Portosystemic shunts have been identified in significant percentage of mice with C57BL/6J background, resulting in major alteration in brain morphometry, brain metabolites, physiological readouts (e.g., body weight, liver enzymes), and cognitive deficits. Prior to study start, plasma bile acid analysis was performed to exclude animals with abnormally high bile acid concentration (>20 μmol/L) which is a surrogate marker of portosystemic liver shunt. The blood samples (100 μl) for bile acid analysis were collected from saphenous vein into pre-cooled (ice bath) Li-Hep-tubes. The tubes were kept on ice and plasma was separated by centrifugation at 2000 g (+4° C.). Approximately 50 μL of plasma from each mouse was aliquoted into pre-cooled polypropylene tubes and stored at −80° C. until being analyzed. Plasma samples were analyzed using Thermofisher Konelab Xti 20 according to manufacturer's instructions. The mice having abnormally high bile acid levels in the plasma (more than 20 μmol/L) were not included in the study.

Body Weights—Body weights were measured at 4 weeks of age (pre-treatment) and twice a week until the end of the study. Terminal body weights were not collected from animals found dead or euthanized moribund.

Rotarod Test—At 10 weeks of age all mice were subjected to rotarod test. One day session included a training trial of 5 min at 4 RPM on the rotarod apparatus (Med Associates Inc, St Albans, VT, USA). One hour later, the mice were tested for 3 consecutive accelerating trials of 6 min. with the speed changing from 0 to 40 RPM over 360 seconds and an inter-trial interval at least 30 min. The latency to fall from the rod was recorded.

Open Field Test—At 12 weeks of age all mice were subjected to open field test. The mice were brought to the experimental room for at least 1 hour acclimation period prior to testing. Activity chambers (Med Associates Inc, St Albans, VT; 27×27×20.3 cm) are equipped with IR beams. Mice were placed in the center of the chamber and their behavior was recorded for 30 min in 5-min bins. Quantitative analysis was performed on the following five dependent measures: total locomotion, locomotion in the center of the open field, rearing rate in the center, total rearing frequency and velocity. The mice were tested at low-stress conditions with light intensity lowered to approximately 10-30 lux of red light.

¹⁸F-FDG-PET Analysis—At 8 and 12 weeks of age, 9 mice per group were fasted overnight (water allowed ad libitum) to standardize the blood glucose level. During this time the mice were held on a warming mat. Before the ¹⁸F-FDG administration, the animals were hydrated with warm saline (10 mL/kg, s.c.). For the PET scan, approximately 15 MBq of ¹⁸F-FDG was administrated i.v. At 20 min post ¹⁸F-FDG-injection, the animals were anesthetized with isoflurane, and PET scan was started at 30 min post injection. A single 15-min PET scan of head area was performed with BioPET small animal PET/CT (Sedecal, Madrid, Spain) followed by CT image of the same region. During the imaging, the temperature and breathing of the animals was monitored (SA Instruments Inc., NY, USA) and maintained at +37° C. and between 70-100 bpm, respectively.

Images were reconstructed with 3DOSEM algorithm with CT-based attenuation correction. Image analysis was performed with PMOD software (v 3.7, PMOD Technologies LLC, Zurich, Switzerland). The ¹⁸F-FDG uptake in brain regions was presented as standardized uptake value (SUV).

Euthanasia & Sample Collection—At 13 weeks of age, 9 mice per group were euthanized by deep anesthesia with sodium pentobarbital (180 mg/kg). The remaining mice were euthanized and any samples were not collected, except the tail and ear samples. A terminal blood sample (as much as possible) was collected by cardiac puncture into a syringe. The whole blood was placed in Li-Hep tube at RT and used immediately for preparation of PBMCs. The whole brain was collected and fresh frozen in liquid nitrogen and stored at −80° C. Blood was collected also from extra animals as staining controls for the flow cytometry assays (n=3 extra animals per day). Tail and ear samples were collected for possible post-study re-genotype analysis.

PBMC Extraction—Whole blood (approximately 400-500 μL) was stored at room temperature (RT) before the start of the PBMC isolations. The PBMCs were isolated using 15-mL Sepmate tubes (SepMate-15 IVD Cat 85415, Stemcell technologies) according to manufacturer's instruction. Sepmate is a tube that facilitates the isolation of PBMCs by density gradient centrifugation using density gradient medium (Lymphoprep, Cat 07801, Stemcell technologies). Blood samples were diluted 1:1 into sterile Dulbecco's Phosphate Buffered Saline with 2% Fetal Bovine Serum (DPBS+2% FBS). 4.5 mL of density gradient medium (Lymphoprep, Cat 07801, Stemcell technologies) was placed into a 15 mL Sepmate tube and the diluted blood sample was added gently by layering over density gradient medium, making sure there was no mixing of layers prior to centrifugation. Samples were centrifuged at 1200 g for 15 minutes at RT (the centrifuge brake should was on). PBMCs were poured from the Sepmate tubes into a new tube. PBMCs were washed with 1 mL sterile DPBS+2% FBS Cat 07905, Stemcell technologies), mixed gently and centrifuged at 300 g for 10 minutes (brake on for pellet formation) at +4° C. Supernatant was removed and 1 mL DPBS+2% FBS was added, cells were resuspended and centrifuged again. Supernatant was carefully removed. The remaining cell pellet was resuspended gently into 250 μL DPBS+2% FBS to get a clear suspension and the tube was put on ice. For cell counting, 10 μL sample was taken and 89 μL DPBS was added. Right before measurement 1 μL of propidium iodide was added and the cell number and viability was determined by MACSquant Analyzer 10 flow cytometer. Appropriate number of cells was aliquoted into mitochondrial membrane potential (JC-1 dye), mitochondrial ROS (MitoSox) and ATP (Cell Titer Glow) analysis.

Biomarker Assays—The PBMCs were processed with JC-1, Mitosox and CellTiter-Glo® 2.0 Assay kit according to manufacturer's instructions.

The cells were analyzed immediately after the staining using the MACSQuant Analyzer 10 (Miltenyi Biotec) and Cytation 3 (Cell titerglow). FlowJo software was used for further analysis of the JC-1 and Mitosox cell populations data. Duplicates were performed when enough sample was available.

TABLE 1
Biomarker and other Reagents

| Reagent | Vendor | CAT | LOT |
|---|---|---|---|
| CellTiter-Glo 2.0 | Promega | G9241 | 0000416139 |
| DPBS | Sigma | D8537 | RNBJ7095 |
| Nunc ™ MicroWell ™ 96-Well, Nunclon | ThermoFisher | 136101 | 162741 |
| Delta-Treated, Flat-Bottom Microplate | | | |
| ATP (Adenosine 5′-triphosphate disodium | Sigma | A7699- | SLCD1217 |
| salt hydrate) | | 1G | |
| MitoSOX Red Reagent | ThermoFisher | M36008 | 2266611 |
| HBSS Ca/Mg free | ThermoFisher | 14025092 | RNBH8767 |
| DMSO | Sigma | D2650 | RNBH3210 |
| DPBS + 2% FBS | Stemcell | 07905 | 1000000000035693 |
| Lymphoprep | Stemcell | 7801 | 00119 |
| Sepmate-15 | Stemcell | 85415 | 206083 |
| Propidium Iodide | Miltenyi | 130-093- | 5210205373 |
| | | 233 | |

Mitochondrial ROS: MitoSOX Assay—MitoSOX™ Red reagent is a novel fluorogenic dye specifically targeted to mitochondria in live cells. Oxidation of MitoSOX™ Red reagent by superoxide produces red fluorescence and can be used to reliably the relative differences in mitochondrial ROS formation in cells. The assay control, heat stressed cells (+63° C. 5 min) mixed with healthy cells 1:1, was used as a positive control for reactive oxygen species formation.

MitoSOX™ Red reagent (Cat M36008 ThermoFisher Scientific) was used according to manufacturer's instructions.

### Protocol in Short:

1. Prepare 5 μM MitoSOX™ reagent working solution. Dilute the 5 mM MitoSOX™ reagent stock solution (prepared above) in HBSS/Ca/Mg or suitable buffer to make a 5 μM MitoSOX™ reagent working solution. Note: The concentration of the MitoSOX™ reagent working solution should not exceed 5 μM. Concentrations exceeding 5 μM can produce cytotoxic effects, including altered mitochondrial morphology and redistribution of fluorescence to nuclei and the cytosol.
2. Place 50,000 cells/well cells in a flow cytometry suitable multiwell plate in DPBS.
3. Spin the cells down 250 G for 5 minutes and remove supernatant.
4. Apply 200 μL of 5 M MitoSOX™ reagent working solution to the cells.
5. Incubate cells for 10 minutes at 37° C., protected from light.
6. Wash cells gently three times with 100 μL of warm DPBS. Spin the cells down 250 G for 5 minutes in each washing step. 7. Immediately collect data: room temperature measurement at dark. Detect ROS signal in the PE channel using a MACSQuant Analyzer 10 (Miltenyi Biotech).

Assay details: The main gating was performed to include all the cell material while leaving out most of the debris. This was further gated by frontal and side scatters to include only singlet cells, leaving out any multi-cell aggregates. For Mitosox positive staining the singlet population was gated for Mitosox red positive cells (red on x-axis) and SSC (Side-scattered light) (on y-axis. The gating was set according to heat stressed cells for the Mitosox red positive gate.

ATP Levels: CellTiter-Glo 2.0® Assay—The CellTiter-Glo® 2.0 Assay provides a homogeneous method to determine the number of viable cells in culture by quantitating the amount of ATP present, which indicates the presence of metabolically active cells.

Cell Viability Assay Cat G9241 Promega was used according to manufacturer's instructions.

### Protocol in Short:

1. Prepare and equilibrate the CellTiter-Glo® 2.0 prior to performing the assay.
2. Place 50,000 cells/well white walled luminescence suitable plate in DPBS.
3. Prepare control wells containing medium without cells to determine background luminescence.
4. Add a volume of CellTiter-Glo® 2.0 Reagent equal to the volume of cell culture medium present in each well (e.g., for a 96-well plate, add 100 μL of CellTiter-Glo® 2.0 Reagent to 100 μL of medium containing cells).
5. Mix the contents for 2 minutes on an orbital shaker to induce cell lysis (see Appendix for more information on mixing).
6. Allow the plate to incubate at room temperature for 10 minutes to stabilize the luminescent signal.
7. Record luminescence with Cytation 3 (BioTek).

Assay details: The plate was placed in Cytation 3 plate reader and the amount of ATP present was determined by reading the chemiluminescence levels in each well. The assay control, heat shocked cells (+63° C. 5 min) mixed with healthy cells 1:1, was used to decrease the number of metabolically active cells (decrease the level of ATP in the heat shocked control cells).

### Statistical Analysis and Data Graphic Presentation

Prior to further statistical analysis, data quality check and validations were performed. During that process, potential outliers will be identified and assessed. No outliers were removed from data without a clear justification for removal (e.g., identified measurement error in the laboratory notes).

The planned comparisons in this study were:
Vehicle treated R6/2 group vs. WT group
Compound (Ia) treated R6/2 groups vs. Vehicle treated R6/2 group

Assumption of normality of each data set was primarily based on experience (e.g., data within a population is known to be approximately Gaussian) and observations during validation phase.

The performed comparisons for the PBMC biomarker analysis in JC1, Mitosox and Cell Titer Glow and in this study were:
Vehicle treated R6/2 group vs. WT group
Compound (Ia) treated R6/2 groups vs. Vehicle treated R6/2 group
Assay control vs. Vehicle treated R6/2 group

Simple comparisons between two groups were performed using unpaired Welch's t-test, or, when the assumption of normality or lognormality is not met, by the Mann-Whitney U-test.

Comparison involving three or more groups were performed using one-way ANOVA followed by Dunnett's multiple comparisons test (treatment vs. control). In case of significantly different SDs detected by Brown-Forsythe test, the assumption of equal SDs was rejected and Welch's ANOVA followed by Dunnett's T3 multiple comparison test was used. The non-parametric counterpart was the Kruskal-Wallis test followed by Dunn's multiple comparison test.

All values are presented as the mean±standard error of the mean. In case of two or more comparisons per family, the multiplicity adjusted P values are presented. All statistical analyses were conducted with a significance level of α=0.05, using GraphPad Prism (Version 8.3, GraphPad Software, Inc., San Diego, CA) or the R statistical software environment (R: A Language and Environment for Statistical Computing; R Core Team/R Foundation for Statistical Computing; Vienna, Austria; 2019; available online at R-project.org).

### Results

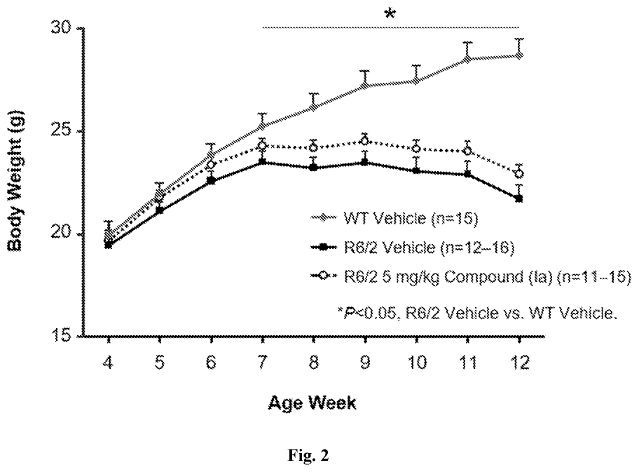

Body Weights—The effects of chronic administration with Compound (Ia) (0.5 or 5 mg/kg, i.p.) on body weight of the wild type and R6/2 mice are presented in FIG. 2. There were no significant differences between the different groups of R6/2 mice in body weight (p>0.05, Mixed-effects model (REML)) (FIG. 2).

Altogether 15 R6/2 mice died before the scheduled end point. For those mice that died before the 12-week imaging new replacement mice were included, starting the dosing at the same age as for the mice initially included in the study.

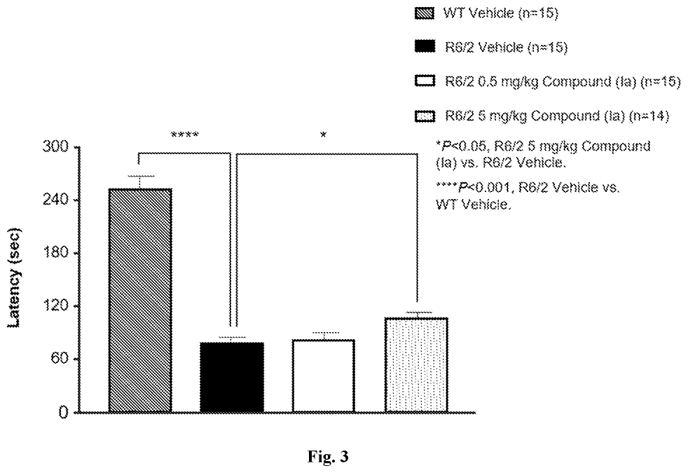

Rotarod—The effects of chronic administration with Compound (Ia) (0.5 or 5 mg/kg, i.p.) on latency to fall in rotarod of the wild type and R6/2 mice at 10 weeks of age are presented in FIG. 3. The R6/2 mice treated with 5 mg/kg of Compound (Ia) had increased latency in the rotarod compared to vehicle treated R6/2 mice (*p<0.05, One-way ANOVA) (FIG. 3).

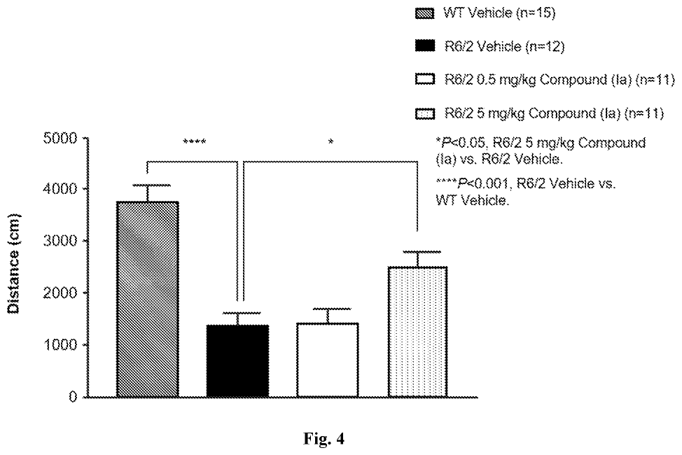

Open Field; TotalDistance Traveled—The effects of chronic administration with Compound (Ia) (0.5 or 5 mg/kg, i.p.) on total distance traveled by the wild type and R6/2 mice in the open field at 12 weeks of age are presented in FIG. 4. The R6/2 mice treated with 5 mg/kg of Compound (Ia) traveled longer total distance compared to vehicle treated R6/2 mice (*p<0.05, One-way ANOVA) (FIG. 4)

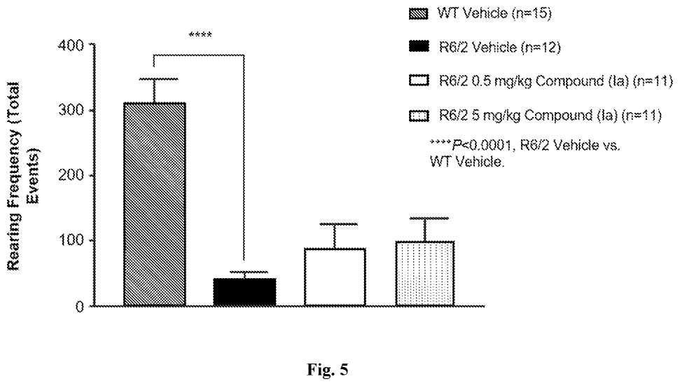

Open Field; Total Rearing Frequency—The effects of chronic administration with Compound (Ia) (0.5 or 5 mg/kg, i.p.) on total rearing frequency of the wild type and R6/2 mice in the open field at 12 weeks of age are presented in FIG. 5. There were no significant differences between the different groups of R6/2 mice in total rearing frequency (p>0.05, One-way ANOVA) (FIG. 5).

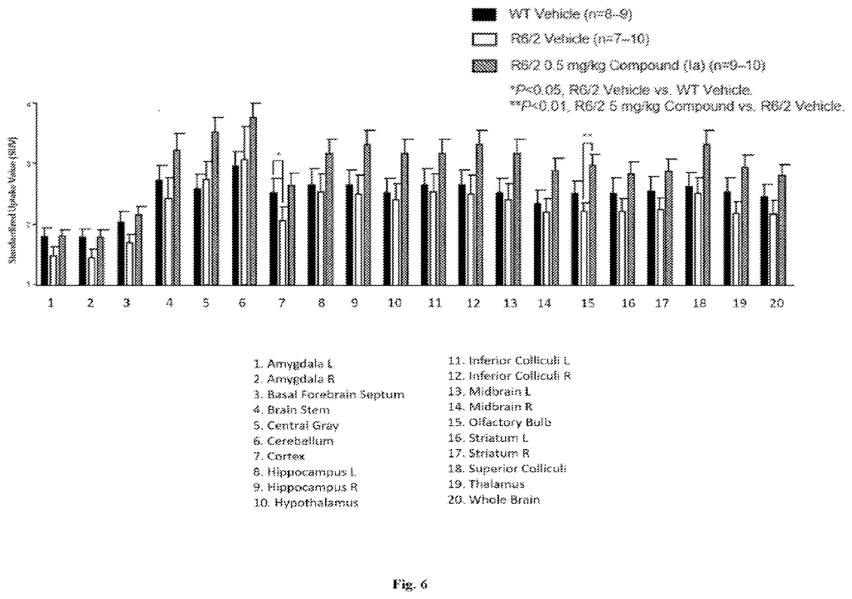

¹⁸F-FDG-PETAssay—A reduction of ¹⁸F-FDG uptake in R6/2 Vehicle group was seen in all brain regions compared to WT Vehicle at 8 weeks of age and at 12 weeks of age in all areas but central grey and cerebellum (FIG. 6). However, the genotype difference was statistically significant only in cortex at 12 weeks of age (p<0.05, Mixed-effects model (REML)) (FIG. 6). Treatment with Compound (Ia) increased the ¹⁸F-FDG uptake in various brain areas in R6/2 mice. However, statistically significantly higher radioactivity was observed only in olfactory bulb at 12 weeks of age in R6/2 mice treated with 5 mg/kg of Compound (Ia) compared to vehicle treated R6/2 mice (p<0.05, Mixed-effects model (REML)) (FIG. 6).

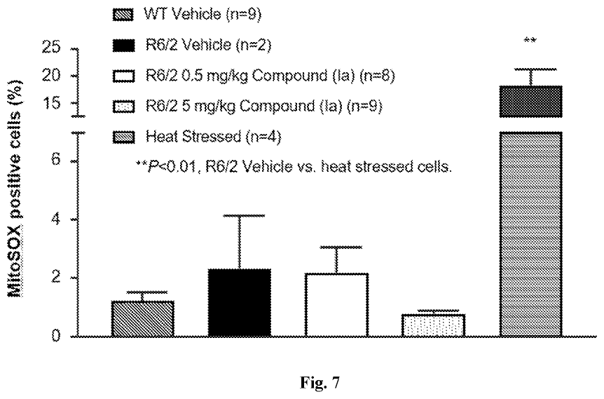

MitoSOXAssay—The Mitosox flow cytometry results showed no significant differences in the ROS formation between WT+Vehicle or R6/2+Vehicle or between R6/2+Vehicle and R6/2+Compound (Ia) (0.5 mg/kg/5 mg/kg) treated groups which indicated that the mitochondrial ROS formation was at similar levels between the groups (FIG. 7). Heat shock induced a significant ROS production in the mitochondria as measured by significant increase in red fluorescent cell population in heat shocked group when compared to R6/2+Vehicle treated mice (FIG. 7).

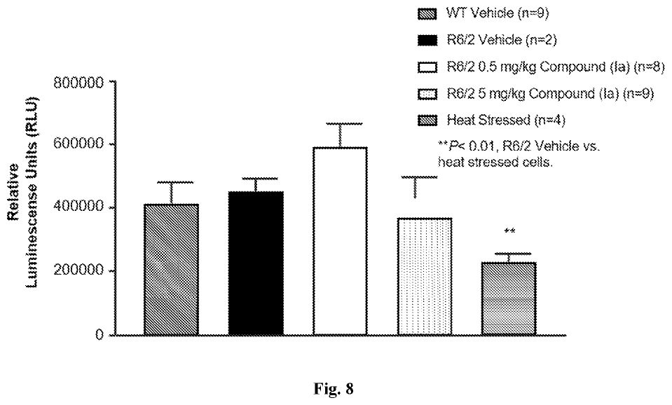

Cell Titer Glow Results (ATP Assay)—The Cell Titer Glow showed no significant differences in the ATP levels between WT+Vehicle and R6/2+Vehicle or between R6/2+Vehicle and R6/2+Compound (Ia) (0.5 mg/kg/5 mg/kg) treated groups, which indicated that ATP levels were the same between the groups (FIG. 8). The assay control, heat stressed cells, showed significant decrease in ATP levels when compared to R6/2+Vehicle treated mice (FIG. 8).

### CONCLUSIONS

The R6/2 mice (Huntington's disease model) were treated once a day with Compound (Ia) (0.5 or 5 mg/kg; i.p., QD) starting from 4 weeks of age. The mice were subjected to various behavioral tests assessing the characteristic motor deficits and progressive phenotype of the R6/2 mice. In addition, the potential effects of Compound (Ia) on factors affecting the brain metabolism were evaluated using ¹⁸F-FDG PET imaging. The body weight of the R6/2 mice was not significantly affected with Compound (Ia) treatment, although the body weight of the mice treated with 5 mg/kg dose remained at a non-significantly higher level compared to vehicle treated R6/2 mice. Similarly, the R6/2 mice treated with the higher, 5 mg/kg dose showed significant improvement in rotarod and increased horizontal activity in the open field at 12 weeks of age. ¹⁸F-FDG PET imaging revealed an increased ¹⁸F-FDG uptake in olfactory bulb at 12 weeks of age. There was a similar trend towards increased ¹⁸F-FDG uptake also in various other brain regions; however, the overall Anova did not reach statistical significance.

The Mitosox flow cytometry results showed no significant differences in the ROS formation between vehicle treated R6/2 mice and wild-type mice, or between the different treatment groups of R6/2 mice, which indicated that the mitochondrial ROS formation was at similar level between the groups. Heat shock induced a significant ROS production in the mitochondria as measured by significant increase in red fluorescent cell population in heat shocked group in vehicle treated R6/2 mice compared to wild-type mice. The Cell Titer Glow showed no significant differences in the ATP levels between the genotypes or the different R6/2 groups, which indicated that ATP levels were the same between the groups. The ATP levels assay control, heat stressed cells showed significant decrease in ATP levels in R6/2 mice compared to wild-type mice.

In summary, the data highlight trends and metabolic biomarker improvements associated with administration of Compound (Ia) to R6/2 mice (a mouse model of Huntington's disease). Accordingly, these results show that compositions comprising Compound (Ia) are useful in methods for treating, preventing, inhibiting, ameliorating, or delaying the onset of Huntington's disease and/or a HTT proteinopathy.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a nonlimiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A method for treating, preventing, inhibiting, ameliorating, or delaying the onset of Huntington's disease and/or a HTT proteinopathy in a subject in need thereof, comprising administering to the subject a therapeutically effective amount of a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

2. The method of claim 1, wherein the subject has been diagnosed as having Huntington's disease.

3. The method of claims 1 or 2, wherein the peptidomimetic is administered to the subject daily for 2 weeks or more, for 12 weeks or more, for 24 weeks or more, for 36 weeks or more, for 48 weeks or more, or for 52 weeks or more.

4. The method of any one of the previous claims, wherein the peptidomimetic is administered daily for the remainder of the life of the subject.

5. The method of any one of the previous claims, wherein the treating, preventing, inhibiting, or ameliorating comprises the treatment, prevention, inhibition, or amelioration of one or more signs or symptoms of Huntington's disease or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

6. The method of any one of the previous claims, wherein the subject is a mammal.

7. The method of claim 6, wherein the mammalian subject is a human.

8. The method of any one of claims 1-7, wherein the peptidomimetic is administered orally.

9. The method of any one of claims 1-7, wherein the peptidomimetic is administered subcutaneously.

10. The method of any one of claims 1-7, wherein the peptidomimetic is administered topically, intranasally, systemically, intravenously, intraperitoneally, intradermally, intraocularly, ophthalmically, intrathecally, intracerebroventricularly, iontophoretically, transmucosally, intravitreally, or intramuscularly.

11. The method of any one of the previous claims, further comprising separately, sequentially, or simultaneously administering an additional treatment to the subject.

12. The method of claim 11, wherein the additional treatment comprises administration of a therapeutic agent.

13. The method of claim 12, wherein the therapeutic agent is selected from the group consisting of: Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine).

14. The method of claim 13, wherein the combination of peptidomimetic and an additional therapeutic treatment has a synergistic effect for the treatment of Huntington's disease and/or HTT proteinopathy.

15. The method of any one of the previous claims, wherein the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

16. The method of any one of claims 1-14, wherein the peptidomimetic is formulated as a tris-HCl salt, a bis-HCl salt, or a mono-HCl salt.

17. Use of a composition in the preparation of a medicament for treating, preventing, inhibiting, ameliorating or delaying the onset of Huntington's disease and/or a HTT proteinopathy in a subject in need thereof, wherein the composition comprises a therapeutically effective amount of a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

18. The use of claim 17, wherein the subject has been diagnosed as having Huntington's disease.

19. The use of claims 17 or 18, wherein the medicament is administered daily for 2 weeks or more, for 12 weeks or more, for 24 weeks or more, for 36 weeks or more, for 48 weeks or more, or for 52 weeks or more.

20. The use of any one of claims 17-19, wherein the medicament is administered daily for the remainder of the life of the subject.

21. The use of any one of claims 17-20, wherein the use of the medicament for the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

22. The use of any one of claims 17-21, wherein the subject is a mammal.

23. The use of claim 22, wherein the mammalian subject is a human.

24. The use of any one of claims 17-23, wherein the medicament is formulated for oral administration.

25. The use of any one of claims 17-23, wherein the medicament is formulated for subcutaneous administration.

26. The use of any one of claims 17-23, wherein the medicament is formulated for administration, topically, intranasally, systemically, intravenously, intraperitoneally, intradermally, intraocularly, ophthalmically, intrathecally, intracerebroventricularly, iontophoretically, transmucosally, intravitreally, or intramuscularly.

27. The use of any one of claims 17-26, wherein the medicament is separately, sequentially, or simultaneously used with an additional treatment.

28. The use of claim 27, wherein the additional treatment comprises use of a therapeutic agent.

29. The use of claim 28, wherein the therapeutic agent is selected from the group consisting of: Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine).

30. The use of any one of claims 27-29, wherein the combination of medicament and an additional treatment has a synergistic effect for the treatment of Huntington's disease and/or HTT proteinopathy.

31. The use of any one of claims 17-30, wherein the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

32. The use of any one of claims 17-30, wherein the peptidomimetic is formulated as a tris-HCl salt, a bis-HCl salt, or a mono-HCl salt.

33. A peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof, for use in treating, preventing, inhibiting, ameliorating and/or delaying the onset of Huntington's disease and/or a HTT proteinopathy in a subject in need thereof.

34. The peptidomimetic of claim 33, for use wherein the subject has been diagnosed as having Huntington's disease.

35. The peptidomimetic of 33 or 34, for use wherein the peptidomimetic is administered to the subject daily for 2 weeks or more, for 24 weeks or more, for 36 weeks or more, for 48 weeks or more, or for 52 weeks or more.

36. The peptidomimetic of 33 or 34, for use wherein the peptidomimetic is administered daily for the remainder of the life of the subject.

37. The peptidomimetic of any one of claims 33-36, for use wherein the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

38. The peptidomimetic of any one of claims 33-37, for use wherein the subject is a mammal.

39. The peptidomimetic of claim 38, for use wherein the mammalian subject is a human.

40. The peptidomimetic of any one of claims 33-39, for use wherein the peptidomimetic is formulated for oral administration.

41. The peptidomimetic of any one of claims 33-39, for use wherein the peptidomimetic is formulated for subcutaneous administration.

42. The peptidomimetic of any one of claims 33-39, for use wherein the peptidomimetic is formulated for administration topically, intranasally, systemically, intravenously, intraperitoneally, intradermally, intraocularly, ophthalmically, intrathecally, intracerebroventricularly, iontophoretically, transmucosally, intravitreally, or intramuscularly.

43. The peptidomimetic of any one of claims 33-42, for use wherein the peptidomimetic is separately, sequentially, or simultaneously used with an additional treatment.

44. The peptidomimetic of claim 43, for use wherein the additional treatment comprises use of a therapeutic agent.

45. The peptidomimetic of claim 44, for use wherein the therapeutic agent is selected from the group consisting of: Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine).

46. The peptidomimetic of any one of claims 33-45, for use wherein the combination of peptidomimetic and an additional treatment has a synergistic effect for the treatment of Huntington's disease and/or HTT proteinopathy.

47. The peptidomimetic of any one of claims 33-46, for use wherein the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

48. The peptidomimetic of any one of claims 33-46, for use wherein the peptidomimetic is formulated as a tris-HCl salt, a bis-HCl salt, or a mono-HCl salt.

49. A formulation or medicament comprising a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof, for use in treating, preventing, inhibiting, ameliorating and/or delaying the onset of Huntington's disease and/or a HTT proteinopathy in a subject in need thereof.

50. The formulation or medicament of claim 49, for use wherein the subject has been diagnosed as having Huntington's disease.

51. The formulation or medicament of 49 or 50, for use wherein the formulation or medicament is administered to the subject daily for 2 weeks or more, for 24 weeks or more, for 36 weeks or more, for 48 weeks or more, or for 52 weeks or more.

52. The formulation or medicament of 49 or 50, for use wherein the formulation or medicament is administered daily for the remainder of the life of the subject.

53. The formulation or medicament of any one of claims 49-52, for use wherein the treating, preventing, inhibiting, or ameliorating comprises the treatment, prevention, inhibition, or amelioration of one or more signs or symptoms of Huntington's disease and/or HTT proteinopathy comprising stumbling, clumsiness, loss of coordination, loss of control of movements, difficulty swallowing, difficulty speaking, loss of ambulation, personality changes, mood changes, apathy, irritability, aggression, anger, depression, frustration, suicidal thoughts, difficulty focusing, lapses in short term memory, loss of initiative, diminishing organizational skills, disorientation, loss of cognitive skills and weight loss.

54. The formulation or medicament of any one of claims 49-53, for use wherein the subject is a mammal.

55. The formulation or medicament of claim 54, for use wherein the mammalian subject is a human.

56. The formulation or medicament of any one of claims 49-55, for use wherein the formulation or medicament is formulated for oral administration.

57. The formulation or medicament of any one of claims 49-55, for use wherein the formulation or medicament is formulated for subcutaneous administration.

58. The formulation or medicament of any one of claims 49-55, for use wherein the formulation or medicament is formulated for administration topically, intranasally, systemically, intravenously, intraperitoneally, intradermally, intraocularly, ophthalmically, intrathecally, intracerebroventricularly, iontophoretically, transmucosally, intravitreally, or intramuscularly.

59. The formulation or medicament of any one of claims 49-58, for use wherein the formulation or medicament is separately, sequentially, or simultaneously used with an additional treatment.

60. The formulation or medicament of claim 59, for use wherein the additional treatment comprises use of a therapeutic agent.

61. The formulation or medicament of claim 60, for use wherein the therapeutic agent is selected from the group consisting of: Xenazine® (tetrabenazine), Austedo® (deutetrabenazine), Risperdal® (risperidone), Haldol® (haloperidol), Thorazine® (chlorpromazine), benzodiazepines, such as Klonopin® (clonazepam) and Valium® (diazepam), Lexapro® (escitalopram), Prozac® (fluoxetine), Zoloft® (sertraline), Seroquel® (quetiapine), Carbatrol® (carbamazepine), Depacon® (valproate sodium), and Lamictal® (lamotrigine).

62. The formulation or medicament of any one of claims 59-61, for use wherein the combination of peptidomimetic and an additional treatment has a synergistic effect for the treatment of Huntington's disease and/or HTT proteinopathy.

63. The formulation or medicament of any one of claims 49-62, for use wherein the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

64. The formulation or medicament of any one of claims 49-62, for use wherein the peptidomimetic is formulated as a tris-HCl salt, a bis-HCl salt, or a mono-HCl salt.
